SEQUENCE LISTING (58 sequences)

SEQ ID NO: 1 - DNA, 20 bp, synthetic construct
  misc_feature 1..20: note = Synthetic: non-targeting (NT) guide (gRNA)
  source 1..20: mol_type = other DNA
gctttcacggaggttcgacg

SEQ ID NO: 2 - DNA, 20 bp, synthetic construct
  misc_feature 1..20: note = Synthetic: ALDH3B2 gRNA
  source 1..20: mol_type = other DNA
gccctcctcacctgcggcga

SEQ ID NO: 3 - DNA, 22 bp, synthetic construct
  misc_feature 1..22: note = Synthetic: ACTB forward primer
  source 1..22: mol_type = other DNA
caccattggcaatgagcggttc

SEQ ID NO: 4 - DNA, 22 bp, synthetic construct
  misc_feature 1..22: note = Synthetic: ACTB reverse primer
  source 1..22: mol_type = other DNA
aggtctttgcggatgtccacgt

SEQ ID NO: 5 - DNA, 22 bp, synthetic construct
  misc_feature 1..22: note = Synthetic: Insulin forward primer
  source 1..22: mol_type = other DNA
acgaggcttcttctacacaccc

SEQ ID NO: 6 - DNA, 22 bp, synthetic construct
  misc_feature 1..22: note = Synthetic: Insulin reverse primer
  source 1..22: mol_type = other DNA
tccacaatgccacgcttctgca

SEQ ID NO: 7 - DNA, 22 bp, synthetic construct
  misc_feature 1..22: note = Synthetic: PDX1 forward primer
  source 1..22: mol_type = other DNA
gaagtctaccaaagctcacgcg

SEQ ID NO: 8 - DNA, 22 bp, synthetic construct
  misc_feature 1..22: note = Synthetic: PDX1 reverse primer
  source 1..22: mol_type = other DNA
ggaactccttctccagctctag

SEQ ID NO: 9 - DNA, 23 bp, synthetic construct
  misc_feature 1..23: note = Synthetic: NKX6.1 forward primer
  source 1..23: mol_type = other DNA
cctattcgttggggatgacagag

SEQ ID NO: 10 - DNA, 22 bp, synthetic construct
  misc_feature 1..22: note = Synthetic: NKX6.1 reverse primer
  source 1..22: mol_type = other DNA
tctgtctccgagtcctgcttct

SEQ ID NO: 11 - DNA, 23 bp, synthetic construct
  misc_feature 1..23: note = Synthetic: PAX6 forward primer
  source 1..23: mol_type = other DNA
ctgaggaatcagagaagacaggc

SEQ ID NO: 12 - DNA, 22 bp, synthetic construct
  misc_feature 1..22: note = Synthetic: PAX6 reverse primer
  source 1..22: mol_type = other DNA
atggagccagatgtgaaggagg

SEQ ID NO: 13 - DNA, 22 bp, synthetic construct
  misc_feature 1..22: note = Synthetic: MAFA forward primer
  source 1..22: mol_type = other DNA
gcttcagcaaggaggaggtcat

SEQ ID NO: 14 - DNA, 22 bp, synthetic construct
  misc_feature 1..22: note = Synthetic: MAFA reverse primer
  source 1..22: mol_type = other DNA
tctggagttggcacttctcgct

SEQ ID NO: 15 - DNA, 23 bp, synthetic construct
  misc_feature 1..23: note = Synthetic: NEUROD forward primer
  source 1..23: mol_type = other DNA
ggtgccttgctattctaagacgc

SEQ ID NO: 16 - DNA, 22 bp, synthetic construct
  misc_feature 1..22: note = Synthetic: NEUROD reverse primer
  source 1..22: mol_type = other DNA
gcaaagcgtctgaacgaaggag

SEQ ID NO: 17 - DNA, 22 bp, synthetic construct
  misc_feature 1..22: note = Synthetic: NGN3 forward primer
  source 1..22: mol_type = other DNA
cctaagagcgagttggcactga

SEQ ID NO: 18 - DNA, 22 bp, synthetic construct
  misc_feature 1..22: note = Synthetic: NGN3 reverse primer
  source 1..22: mol_type = other DNA
agtgccgagttgaggttgtgca

SEQ ID NO: 19 - DNA, 22 bp, synthetic construct
  misc_feature 1..22: note = Synthetic: GCG forward primer
  source 1..22: mol_type = other DNA
cgttcccttcaagacacagagg

SEQ ID NO: 20 - DNA, 22 bp, synthetic construct
  misc_feature 1..22: note = Synthetic: GCG reverse primer
  source 1..22: mol_type = other DNA
acgcctggagtccagatacttg

SEQ ID NO: 21 - DNA, 22 bp, synthetic construct
  misc_feature 1..22: note = Synthetic: SST forward primer
  source 1..22: mol_type = other DNA
ccagactccgtcagtttctgca

SEQ ID NO: 22 - DNA, 22 bp, synthetic construct
  misc_feature 1..22: note = Synthetic: SST reverse primer
  source 1..22: mol_type = other DNA
ttccagggcatcattctccgtc

SEQ ID NO: 23 - DNA, 22 bp, synthetic construct
  misc_feature 1..22: note = Synthetic: PP forward primer
  source 1..22: mol_type = other DNA
agacacaaagaggacacgctgg

SEQ ID NO: 24 - DNA, 22 bp, synthetic construct
  misc_feature 1..22: note = Synthetic: PP reverse primer
  source 1..22: mol_type = other DNA
gagtcgtaggagacagaaggtg

SEQ ID NO: 25 - DNA, 22 bp, synthetic construct
  misc_feature 1..22: note = Synthetic: SLC2A2 forward primer
  source 1..22: mol_type = other DNA
atgtcagtgggacttgtgctgc

SEQ ID NO: 26 - DNA, 22 bp, synthetic construct
  misc_feature 1..22: note = Synthetic: SLC2A2 reverse primer
  source 1..22: mol_type = other DNA
aactcagccaccatgaaccagg

SEQ ID NO: 27 - DNA, 22 bp, synthetic construct
  misc_feature 1..22: note = Synthetic: GCK forward primer
  source 1..22: mol_type = other DNA
catctccgacttcctggacaag

SEQ ID NO: 28 - DNA, 22 bp, synthetic construct
  misc_feature 1..22: note = Synthetic: GCK reverse primer
  source 1..22: mol_type = other DNA
tggtccagttgagaaggatgcc

SEQ ID NO: 29 - DNA, 22 bp, synthetic construct
  misc_feature 1..22: note = Synthetic: ABCC8 forward primer
  source 1..22: mol_type = other DNA
gacgacaagaggacagtggtct

SEQ ID NO: 30 - DNA, 22 bp, synthetic construct
  misc_feature 1..22: note = Synthetic: ABCC8 reverse primer
  source 1..22: mol_type = other DNA
gcattcagacctctggaagtcc

SEQ ID NO: 31 - DNA, 22 bp, synthetic construct
  misc_feature 1..22: note = Synthetic: KCNJ11 forward primer
  source 1..22: mol_type = other DNA
tgtgtcaccagcatccactcct

SEQ ID NO: 32 - DNA, 22 bp, synthetic construct
  misc_feature 1..22: note = Synthetic: KCNJ11 reverse primer
  source 1..22: mol_type = other DNA
gttctgcacgatgaggatcagg

SEQ ID NO: 33 - DNA, 22 bp, synthetic construct
  misc_feature 1..22: note = Synthetic: IA2 forward primer
  source 1..22: mol_type = other DNA
tggagatcctggctgagcatgt

SEQ ID NO: 34 - DNA, 22 bp, synthetic construct
  misc_feature 1..22: note = Synthetic: IA2 reverse primer
  source 1..22: mol_type = other DNA
ggtcacatcagccaaagacagg

SEQ ID NO: 35 - DNA, 22 bp, synthetic construct
  misc_feature 1..22: note = Synthetic: KRT19 forward primer
  source 1..22: mol_type = other DNA
agctagaggtgaagatccgcga

SEQ ID NO: 36 - DNA, 22 bp, synthetic construct
  misc_feature 1..22: note = Synthetic: KRT19 reverse primer
  source 1..22: mol_type = other DNA
gcaggacaatcctggagttctc

SEQ ID NO: 37 - DNA, 22 bp, synthetic construct
  misc_feature 1..22: note = Synthetic: CA2 forward primer
  source 1..22: mol_type = other DNA
gtgacctggattgtgctcaagg

SEQ ID NO: 38 - DNA, 23 bp, synthetic construct
  misc_feature 1..23: note = Synthetic: CA2 reverse primer
  source 1..23: mol_type = other DNA
gttgtccaccatcagttcttcgg

SEQ ID NO: 39 - DNA, 22 bp, synthetic construct
  misc_feature 1..22: note = Synthetic: HNF1B forward primer
  source 1..22: mol_type = other DNA
cccagcaaatcttgtaccaggc

SEQ ID NO: 40 - DNA, 22 bp, synthetic construct
  misc_feature 1..22: note = Synthetic: HNF1B reverse primer
  source 1..22: mol_type = other DNA
acctcagtgaccaagttggagc

SEQ ID NO: 41 - DNA, 21 bp, synthetic construct
  misc_feature 1..21: note = Synthetic: SOX9 forward primer
  source 1..21: mol_type = other DNA
aggaagctcgcggaccagtac

SEQ ID NO: 42 - DNA, 22 bp, synthetic construct
  misc_feature 1..22: note = Synthetic: SOX9 reverse primer
  source 1..22: mol_type = other DNA
ggtggtccttcttgtgctgcac

SEQ ID NO: 43 - DNA, 31 bp, synthetic construct
  misc_feature 1..31: note = Synthetic: Human insulin promoter forward primer
  source 1..31: mol_type = other DNA
aggataggttgtattagaagaggttattaag

SEQ ID NO: 44 - DNA, 26 bp, synthetic construct
  misc_feature 1..26: note = Synthetic: Human insulin promoter reverse primer
  source 1..26: mol_type = other DNA
cccctaaactcacccccacatacttc

SEQ ID NO: 45 - DNA, 62 bp, synthetic construct
  misc_feature 1..62: note = Synthetic: Sequence flanking the gRNA targeting site of wildtype ALDH3B2
  source 1..62: mol_type = other DNA
cggggagagcatggggttcggaacgccctcctcacctgcggcgagggcgcccaccaggagca

SEQ ID NO: 46 - DNA, 59 bp, synthetic construct
  misc_feature 1..59: note = Synthetic: Sequence of ALDH3B2 indel mutant 1 (Mut1)
  source 1..59: mol_type = other DNA
cggggagagcatggggttcggaacgccctcctcacctgcgagggcgcccaccaggagca

SEQ ID NO: 47 - DNA, 63 bp, synthetic construct
  misc_feature 1..63: note = Synthetic: Sequence of ALDH3B2 indel mutant 2 (Mut)
  source 1..63: mol_type = other DNA
cggggagagcatggggttcggaacgccctcctcacctggcggcgagggcgcccaccaggagca

SEQ ID NO: 48 - DNA, 53 bp, synthetic construct
  misc_feature 1..53: note = Synthetic: Sequence of ALDH3B2 indel mutant 3 (Mut3)
  source 1..53: mol_type = other DNA
cggggagagcatggggttcggaacgccctcctcacctgcgcccaccaggagca

SEQ ID NO: 49 - DNA, 33 bp, synthetic construct
  misc_feature 1..33: note = Synthetic: Sequence of ALDH3B2 indel mutant 4 (Mut4)
  source 1..33: mol_type = other DNA
cggggagagcatggggttcggaacgcaggagca

SEQ ID NO: 50 - DNA, 37 bp, synthetic construct
  misc_feature 1..37: note = Synthetic: Sequence of ALDH3B2 indel mutant 5 (Mut5)
  source 1..37: mol_type = other DNA
cggggagagcatggggttcggaaccccaccaggagca

SEQ ID NO: 51 - DNA, 64 bp, synthetic construct
  misc_feature 1..64: note = Synthetic: Sequence of ALDH3B2 indel mutant 6 (Mut6)
  source 1..64: mol_type = other DNA
cggggagagcatggggttcggaacgccctcctcacctgcggcgcgagggcgcccaccaggagca

SEQ ID NO: 52 - DNA, 56 bp, synthetic construct
  misc_feature 1..56: note = Synthetic: Sequence of ALDH3B2 indel mutant 7 (Mut7)
  source 1..56: mol_type = other DNA
cggggagagcatggggttcggaacgccctcctcacctgcggcgcccaccaggagca

SEQ ID NO: 53 - DNA, 50 bp, synthetic construct
  misc_feature 1..50: note = Synthetic: Sequence of ALDH3B2 indel mutant 8 (Mut8)
  source 1..50: mol_type = other DNA
cggggagagcatggggttcggaacgccctcctcacctgcggcgaggagca

SEQ ID NO: 54 - DNA, 63 bp, synthetic construct
  misc_feature 1..63: note = Synthetic: Sequence of ALDH3B2 indel mutant 9 (Mut9)
  source 1..63: mol_type = other DNA
cggggagagcatggggttcggaacgccctcctcacctgccggcgagggcgcccaccaggagca

SEQ ID NO: 55 - DNA, 59 bp, synthetic construct
  misc_feature 1..59: note = Synthetic: Sequence of ALDH3B2 indel mutant 10 (Mut10)
  source 1..59: mol_type = other DNA
cggggagagcatggggttcggaacgccctcctcacctgcggaggcgcccaccaggagca

SEQ ID NO: 56 - DNA, 53 bp, synthetic construct
  misc_feature 1..53: note = Synthetic: Sequence of ALDH3B2 indel mutant 11 (Mut11)
  source 1..53: mol_type = other DNA
cggggagagcatggggttcggaacgccctccctgagggcgcccaccaggagca

SEQ ID NO: 57 - DNA, 57 bp, synthetic construct
  misc_feature 1..57: note = Synthetic: Sequence of ALDH3B2 indel mutant 12 (Mut12)
  source 1..57: mol_type = other DNA
cggggagagcatggggttcggaacgccctcctcacctgcgggcgcccaccaggagca

SEQ ID NO: 58 - DNA, 43 bp, synthetic construct
  misc_feature 1..43: note = Synthetic: Sequence of ALDH3B2 indel mutant 13 (Mut13)
  source 1..43: mol_type = other DNA
cggggagagcatggggttcggaacgccctcctcaccaggagca

(19) United States
(12) Patent Application Publication
(10) Pub. No.: US 2025/0144152 A1
(43) Pub. Date: May 8, 2025

(54) Transdifferentiation of Pancreatic Duct Cells Into Beta-Like Cells

(71) Applicant: Joslin Diabetes Center, Boston, MA (US)

(72) Inventor: Peng Yi, Needham, MA (US)

(73) Assignee: Joslin Diabetes Center, Boston, MA (US)

(21) Appl. No.: 18/978,242

(22) Filed: Dec. 12, 2024

Related U.S. Application Data
(63) Continuation of application No. PCT/US23/68346, filed on Jun. 13, 2023.
(60) Provisional application No. 63/352,078, filed on Jun. 14, 2022.

(51) Int. Cl. A61K 35/39; C12N 9/02; C12N 9/22; C12N 15/113; C12N 15/90

(52) U.S. Cl. CPC A61K 35/39; C12N 9/0008; C12N 9/22; C12N 15/1137; C12N 15/907; C12Y 102/01003; C12N 2310/20

(57) ABSTRACT

This disclosure relates to methods of inducing insulin production from pancreatic duct cells in response to glucose or transdifferentiating pancreatic duct cells into insulin-producing beta-like cells by administering an agent that inhibits aldehyde dehydrogenase family 3 member B2 (ALDH3B2) protein or an agent that inhibits the expression of ALDH3B2 gene. This disclosure also relates to methods of treatment of diabetes by administering an agent that inhibits ALDH3B2 protein or an agent that inhibits the expression of ALDH3B2 gene.

This application claims priority to U.S. Provisional Application No. 63/352,078 filed Jun. 14, 2022, which is incorporated herein in its entirety for any purpose.

### SUPPORT

Supported by the Thomas J. Beatson Jr. Foundation Grant #2019-011.

### REFERENCE TO SEQUENCE LISTING

The present application contains a Sequence Listing which has been submitted electronically in XML format. Said XML copy, created on Jun. 12, 2023, is named “01123-0012-00PCT-ST26” and is 75,786 bytes in size. The information in the electronic format of the sequence listing is incorporated herein by reference in its entirety.

### FIELD

This disclosure relates to methods of inducing insulin production from pancreatic duct cells in response to glucose or transdifferentiating pancreatic duct cells into insulin-producing beta-like cells by administering an agent that inhibits aldehyde dehydrogenase family 3 member B2 (ALDH3B2) protein or an agent that inhibits the expression of ALDH3B2 gene. This disclosure also relates to methods of treatment of diabetes by administering an agent that inhibits ALDH3B2 protein or an agent that inhibits the expression of ALDH3B2 gene.

### BACKGROUND

Diabetes, no matter the cause or type, is a disease of complete or relative deficiency of functional pancreatic beta cell mass 1. Therefore, restoration of function pancreatic beta cell mass is critical to a cure for both type 1 and type 2 diabetes. Beta cell mass can be replenished by transplanting of exogenous human cadaver islets or human embryonic stem cells (hESC)/induced pluripotent stem cells (iPSCs) derived beta-like cells²⁻⁵, or alternatively, by promoting endogenous beta cell regeneration. In most cases, transplanted beta cells/islets are HLA-mismatched with the recipient patients, and immunosuppressant needs to be used to prevent graft rejection. On the other hand, promoting the regeneration of a patient's own beta cells bypasses the problem of HLA matching and hence does not require immunosuppressant, and therefore it is an attractive strategy for beta cell mass replenishment. Pancreatic beta cells regeneration can be achieved by beta cell self-duplication⁶ or beta cell transdifferentiation from other pancreatic cell types such as pancreatic alpha cells^{7,8}, acinar cells⁹ or duct cells (reviewed in¹⁰). It has been long believed that pancreatic duct cells may serve as a pool of progenitors for both the islet and acinar tissues after birth and into adulthood, and beta cell transdifferentiation from pancreatic duct cells is also called beta cell neogenesis¹¹⁻¹³. Studies using rodent models showed that pancreatic beta cell replication is the dominant mechanism of beta cell regeneration, but in human, beta cell replication rate is extremely low and it is believed that human beta cells regeneration is achieved mainly by neogenesis¹⁴. Although human beta cell neogenesis is frequently observed evidenced by existence of insulin expressing cells in pancreatic duct epithelium, questions remain as exactly how beta cell neogenesis is controlled and whether beta cell neogenesis from pancreatic duct cells can be induced with high efficiency to replenish functional beta cell mass.

Described herein is a genome-wide CRISPR screen to dissect the mechanism of human beta cell neogenesis and look for therapeutic targets to promote beta cell neogenesis for beta cell regeneration. These experiments highlight inhibition of aldehyde dehydrogenase family 3 member B2 (ALDH3B2) as a means to increase insulin production from pancreatic duct cells in response to glucose.

### SUMMARY

In accordance with the description, this disclosure describes methods of transdifferentiating pancreatic duct cells into insulin-producing beta-like by administering an agent that inhibits ALDH3B2 protein or an agent that inhibits the expression of ALDH3B2 gene. Also described herein of methods of treating diabetes.

Embodiment 1. A method of inducing insulin production from pancreatic duct cells in response to glucose comprising administering an agent that inhibits ALDH3B2 protein or an agent that inhibits the expression of ALDH3B2 gene.

Embodiment 2. The method of embodiment 1, wherein the insulin production in response to glucose is maintained after withdrawing the agent.

Embodiment 3. A method of transdifferentiating pancreatic duct cells into insulin-producing beta-like cells comprising administering an agent that inhibits ALDH3B2 protein or an agent that inhibits the expression of ALDH3B2 gene.

Embodiment 4. The method of embodiment 3, wherein the efficiency of transdifferentiating is 5% or more, 10% or more, 15% or more, 20% or more, or 25% or more.

Embodiment 5. The method of embodiment 3 or embodiment 4, wherein the insulin-producing beta-like cells are maintained after withdrawing the agent.

Embodiment 6. The method of any one of embodiments 3-5, wherein the insulin-producing beta-like cells comprise insulin granules.

Embodiment 7. The method of any one of embodiments 1-6, wherein administration of the agent results in epigenetic changes.

Embodiment 8. The method of embodiment 7, wherein the epigenetic change is a reduction in DNA methylation in the insulin gene locus.

Embodiment 9. A method of treating a subject with diabetes comprising administering an agent that inhibits ALDH3B2 protein or an agent that inhibits the expression of ALDH3B2 gene, wherein the agent increases insulin secretion from the pancreas.

Embodiment 10. The method of embodiment 9, wherein the insulin secretion is from pancreatic duct cells or insulin-producing beta-like cells transdifferentiated from pancreatic duct cells.

Embodiment 11. The method of embodiment 9 or embodiment 10, wherein the subject has a blood sugar level higher than 11.1 mmol/liter or 200 mg/dl.

Embodiment 12. The method of any one of embodiments 9-11, wherein the subject has autoimmune diabetes.

Embodiment 13. The method of embodiment 12, wherein the subject has type 1 diabetes or autoimmune diabetes induced by an immunotherapy.

Embodiment 14. The method of any one of embodiments 9-11, wherein the subject has type 2 diabetes and/or insulin resistance.

Embodiment 15. The method of any one of embodiments 9-14, wherein the subject has lower baseline blood glucose after administration.

Embodiment 16. The method of any one of embodiments 9-15, wherein the subject has lower blood glucose in response to a glucose challenge after administration.

Embodiment 17. A method of preventing the development of diabetes comprising:
a. screening a subject for risk factors for developing diabetes;
b. determining if the subject has increased risk of developing diabetes; and
c. administering an agent that inhibits ALDH3B2 protein or an agent that inhibits the expression of ALDH3B2 gene to a subject that has an increased risk of diabetes.

Embodiment 18. The method of embodiment 17, wherein screening a subject for risk factors comprises obtaining data on a genetic risk score that is based on the known diabetes-associated gene variants, a family history of diabetes, the presence of one or more autoantibodies against beta cell antigens that are known to predict disease risk, and/or abnormal glucose tolerance.

Embodiment 19. The method of any one of embodiments 9-18, wherein the subject is a mammal.

Embodiment 20. The method of embodiment 19, wherein the subject is human.

Embodiment 21. The method of any one of embodiments 1-20, wherein the agent is one or more inhibitor of the ALDH3B2 protein.

Embodiment 22. The method of embodiment 21, wherein the agent is an ALDH inhibitor.

Embodiment 23. The method of embodiment 21 or 22, wherein the inhibitor is 4-amino-4-methyl-2-pentyne-1-al, benomyl, chloral hydrate, chlorpropamide analogs, citral, coprine, cyanamide, daidzin, 4-(diethylamino)benzaldehyde, disulfiram, gossypol, kynurenine metabolites, molinate, nitroglycerin, and/or pargyline.

Embodiment 24. The method of any one of embodiments 1-23, wherein the agent decreases or eliminates expression of the ALDH3B2 protein.

Embodiment 25. The method of embodiment 24, wherein the agent is a gene editing tool.

Embodiment 26. The method of embodiment 25, wherein the gene editing tool is a CRISPR/Cas system, transcription activator-like effector nuclease (TALEN), homing endonuclease, or zinc-finger nuclease (ZFN).

Embodiment 27. The method of embodiment 26, wherein the gene editing tool is delivered by virus transduction or lipid nanoparticle delivery.

Embodiment 28. The method of embodiment 24, wherein the agent is a small interfering RNA.

Embodiment 29. The method of any one of embodiments 9-28, wherein the agent is administered in combination with an additional treatment.

Embodiment 30. The method of embodiment 29, wherein the additional treatment is a glucagon-like peptide analog or agonist, dipeptidyl peptidase-4 inhibitor, amylin analog, biguanide, thiazolidinedione, sulfonylurea, meglitinide, alpha-glucosidase inhibitor, or sodium/glucose transporter 2 inhibitor.

Additional objects and advantages will be set forth in part in the description which follows, and in part will be understood from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIGS. 1A-1D show a summary of a genome-wide CRISPR screen that identified ALDH3B2 as a regulator of human beta cell neogenesis. (A) The structure of REPB reporter. The REPB reporter contains Rat insulin promoter (RIP3.1) driving the expression of EGFP and Blasticidin-S deaminase (BSD), wherein the EGFP and BSD genes are fused together with P2A peptide. (B) An illustration of genome-wide CRISPR screen workflow. (C) gRNA profile volcano plots of the genome-wide CRISPR screen showing the depleted and enriched gRNA in the EGFPhigh, blasticidin resistance REPB-PANC-1 cells. The x-axis is the fold change of gRNA counts (read per million, CPM), and the y-axis is statistical significance as shown by −log 10 of the false discovery rate corrected p value. The vertical dashed line represents a fold change for gene threshold of 1, the horizontal dashed line represents a p value threshold of 0.05. Several highly enriched gRNAs are labeled as large dots. (D) qPCR analysis of INS and KRT19 expression level for top 9 candidate gene mutant PANC-1 cells.

FIGS. 2A-2O show that mutation of ALDH3B2 transdifferentiates PANC-1 cells into insulin-producing beta-like cells. (A) Generation of non-targeting (REPB-NTC, non-targeting control) and REPB-ALDH3B2mut PANC-1 cells. (B) ALDH3B2 expression is substantially reduced in REPB-ALDH3B2mut cells compared to REPB-NTC cells, as shown by western blot. (C) Quantification of Western blot data. Images were obtained and quantified using a C-DiGit scanner and the Image Studio software (LI-COR Biosciences). n=3 per group. Data show mean±SEM, **P<0.01. (D) Insulin and C-peptide immunofluorescence in NTC cells and ALDH3B2mut PANC-1 cells. (E) Insulin content in NTC cells and ALDH3B2mut PANC-1 cells were measured by insulin ELISA, normalized by cells genomic DNA content. n=7 technical replicates per condition and genotype. Data show mean±SEM, **p<0.01. qPCR analysis of human beta cell and duct signature genes was also analyzed. Data show mean±SEM of n=4 replicates per condition and are representative of 2-3 independent experiments. *p<0.05, **p<0.01, calculated by two-way ANOVA with Sidak's multiple comparisons test. Gene expression studies were also performed. (F) Genes related to beta cell key transcription factors. (G) Genes related to pancreatic endocrine hormones. (H) Genes related to pancreatic duct cell markers. (I) Genes related to beta cell function genes. (J) Electron microscopy (EM) analysis of REPB-NTC cells and REPB-ALDH3B2mut cells. (K) Quantification of insulin granules per cell. (L) An illustration of the in vivo functional evaluation of transdifferentiated PANC-1 cells. (M) An intraperitoneal glucose tolerance test (IPGTT) in NTC or ALDH3B2mut PANC-1 cells transplanted NSG mice 6 weeks post-transplantation. (N) Random blood glucose levels in the NTC and ALDH3B2mut transplanted diabetic NSG mice over 8 weeks of monitoring. Data show mean±SEM of n=5 mice (NTC) and n=6 mice (ALDH3B2mut) per group and are representative of three experiments, calculated by two-way ANOVA with Tukey's multiple comparisons test. (0) Serum human insulin levels measurement in the NTC and ALDH3B2mut transplanted diabetic NSG mice.

FIGS. 3A-3J show that knock-down of ALDH3B2 by short hairpin RNA (shRNA) transdifferentiates PANC-1 cells into beta-like cells. (A) Generation of inducible non-targeting (shControl) cells and shALDH3B2 PANC-1 cells. (B) ALDH3B2 expression is significantly reduced in shALDH3B2 PANC-1 cells with doxycycline treatment shown by western blot. (C) Quantification of Western blot data. Images were obtained and quantified using a C-DiGit scanner and the Image Studio software (LI-COR Biosciences). n=2 per group and are representative of two independent experiments, calculated by one-way ANOVA with Dunnett's multiple comparisons test. *shControl vs. shALDH3B2 cells in non-treatment group; #shControl vs. shALDH3B2 in Dox-treatment group. (D) C-peptide immunofluorescence imaging in shControl and shALDH3B2 PANC-1 cells with doxycycline treatment. qPCR analysis of human beta cell and duct signature genes was also performed. Data show mean±SEM of n=4 technical replicates per condition and are representative of 2-3 independent experiments. *p<0.05, **p<0.01, calculated by two-way ANOVA with Sidak's multiple comparisons test. (E) Genes related to beta cell key transcription factors. (F) Genes related to pancreatic endocrine hormones. (G) Genes related to beta cell function genes. (H) Genes related to pancreatic duct cell markers. (I) An illustration of the doxycycline withdraw study in the inducible shControl and shALDH3B2 PANC-1 cells. (J) qPCR analysis of pancreatic beta cell or duct cell signature genes. n=4 per group and are representative of two independent experiments, calculated by two-way ANOVA with Sidak's multiple comparisons test. *shControl(+Dox) vs. shALDH3B2(+Dox 7d) cells; #shControl(+Dox) vs. shALDH3B2(+Dox 7d, −Dox 5d).

FIGS. 4A-4I show disruption of ALDH3B2 transdifferentiates human primary pancreatic ductal cells into beta-like cells. (A) Generation of non-targeting control (NTC) and ALDH3B2mut human primary pancreatic duct cells (HPPD). qPCR analysis of was also performed of human insulin (B), KRT19 (C) and ALDH3B2 (D) expression in purified human primary pancreatic duct cells (HPPD) and human primary islets. Data show mean±SEM of n=3. ***p<0.005. (E), Insulin and CK19 immunofluorescence in HPPD-NTC and HPPD-ALDH3B2mut cells. qPCR analysis of human beta cell and duct signature genes was also performed. Data show mean±SEM of n=3 technical replicates per condition and are representative of 2 independent experiments. *P<0.05, **P<0.01, calculated by two-way ANOVA with Sidak's multiple comparisons test. (F) Genes related to beta cell function genes and key transcription factors. (G) Genes related to pancreatic endocrine hormones. (H) Genes related to beta cell function genes. (I) Genes related to pancreatic duct cell markers.

FIGS. 5A-5D show ALDH3B2 mutation transdifferentiated human primary pancreatic duct cells are functional in vivo. (A) An illustration of the in vivo function evaluation of HPPD-NTC cells and HPPD-ALDH3B2mut cells. (B) Random blood glucose levels in the HPPD-NTC cells and HPPD-ALDH3B2mut cells transplanted diabetic NSG mice over 3 weeks of monitoring. Data show mean±SEM of n=5 mice per group, calculated by two-way ANOVA with Tukey's multiple comparisons test. Serum human insulin levels in non-transplanted NSG mice (Normal NSG), HPPD-NTC cells and HPPD-ALDH3B2mut cells transplanted NSG mice 5 minutes after intraperitoneal (IP) glucose injection were evaluated at 1 week post-transplantation (C) and 3 weeks post-transplantation (D). Calculated by two-way ANOVA with Sidak's multiple comparisons test. * Normal NSG vs. HPPD-NTC; #Normal NSG vs. HPPD-ALDH3B2mut. (E) Insulin, CK19, and Cas9 (Flag) immunofluorescence of transplanted HPPD-NTC and HPPD-ALDH3B2mut cells. (F) Pdx1, CK19 and Cas9 (Flag) immunofluorescence of transplanted HPPD-NTC and HPPD-ALDH3B2mut cells. The percentage of Cas9 lentivirus transduced cells were measured for all transplanted duct cells (G), Pdx1+ cells in all Cas9 lentivirus transduced cells (H), Insulin+ cells in all Cas9 lentivirus transduced cells (I) and Insulin+ cells in all Pdx1+ cells (J).

FIGS. 6A-6E show ALDH3B2 loss-of-function in pancreatic duct cells leads to epigenetic changes. (A) An illustration of the DNA methylation analysis for NTC or ALDH3B2mut pancreatic duct cells. (B) Pattern and percentage of the DNA methylation at the position +63, +127 and +139 of the human insulin gene locus in NTC or ALDH3B2mut PANC-1 cells. (C) Overall quantification of the DNA methylation percentage in panel B. (D) Pattern and percentage of the DNA methylation at the position +63, +127 and +139 of the human insulin gene locus in HPPD-NTC, HPPD-ALDH3B2mut and human primary pancreatic islet cells. (E) overall quantification of the DNA methylation percentage in panel D.

FIGS. 7A and 7B show characterization of the REPB reporter. EGFP expression by fluorescent imaging (A) and quantification of EGFP intensity (B) of the REPB reporter lentivirus infected PANC-1 and NIT-1 cells.

FIG. 8 shows flow cytometric sorting of the GFPhigh PANC-1 cells. Flow cytometry gating strategy of the genome-wide CRISPR screen. P6 population is considered as GFPhigh cells and isolated for subsequent sequencing.

FIGS. 9A and 9B show quantification of GCG and SST expression. qPCR analysis of Glucagon (GCG, A) and Somatostatin (SST, B) expression level for top 9 candidate gene mutant PANC-1 cells.

FIG. 10 shows indel analysis of the ALDH3B2mut PANC-1 cells. The genome sequence flanking the gRNA targeting sites were sequenced and the most abundant indel mutations and their frequency is listed. The ALDH3B2 gRNA targeting site is labelled in bold font. “WT” and “MUT1” through “MUT13” correspond to SEQ ID NOs: 45-58, respectively.

### DESCRIPTION OF THE SEQUENCES

TABLE 1
Table 1 provides a listing of certain
sequences referenced herein.
Description of the Sequences

| | | SEQ |
|---|---|---|
| | | ID |
| Description | Sequences | NO |
| non- | GCTTTCACGGAGGTTCGACG | 1 |
| targeting | | |
| (NT) | | |
| guide | | |
| (gRNA) | | |
| ALDH3B | GCCCTCCTCACCTGCGGCGA | 2 |
| 2 gRNA | | |
| ACTB | CACCATTGGCAATGAGCGGTTC | 3 |
| forward | | |
| primer | | |
| ACTB | AGGTCTTTGCGGATGTCCACGT | 4 |
| reverse | | |
| primer | | |
| Insulin | ACGAGGCTTCTTCTACACACCC | 5 |
| forward | | |
| primer | | |
| Insulin | TCCACAATGCCACGCTTCTGCA | 6 |
| reverse | | |
| primer | | |
| PDX1 | GAAGTCTACCAAAGCTCACGCG | 7 |
| forward | | |
| primer | | |
| PDX1 | GGAACTCCTTCTCCAGCTCTAG | 8 |
| reverse | | |
| primer | | |
| NKX6.1 | CCTATTCGTTGGGGATGACAGAG | 9 |
| forward | | |
| primer | | |
| NKX6.1 | TCTGTCTCCGAGTCCTGCTTCT | 10 |
| reverse | | |
| primer | | |
| PAX6 | CTGAGGAATCAGAGAAGACAGGC | 11 |
| forward | | |
| primer | | |
| PAX6 | ATGGAGCCAGATGTGAAGGAGG | 12 |
| reverse | | |
| primer | | |
| MAFA | GCTTCAGCAAGGAGGAGGTCAT | 13 |
| forward | | |
| primer | | |
| MAFA | TCTGGAGTTGGCACTTCTCGCT | 14 |
| reverse | | |
| primer | | |
| NEURO | GGTGCCTTGCTATTCTAAGACGC | 15 |
| D | | |
| forward | | |
| primer | | |
| NEURO | GCAAAGCGTCTGAACGAAGGAG | 16 |
| D reverse | | |
| primer | | |
| NGN3 | CCTAAGAGCGAGTTGGCACTGA | 17 |
| forward | | |
| primer | | |
| NGN3 | AGTGCCGAGTTGAGGTTGTGCA | 18 |
| reverse | | |
| primer | | |
| GCG | CGTTCCCTTCAAGACACAGAGG | 19 |
| forward | | |
| primer | | |
| GCG | ACGCCTGGAGTCCAGATACTTG | 20 |
| reverse | | |
| primer | | |
| SST | CCAGACTCCGTCAGTTTCTGCA | 21 |
| forward | | |
| primer | | |
| SST | TTCCAGGGCATCATTCTCCGTC | 22 |
| reverse | | |
| primer | | |
| PP | AGACACAAAGAGGACACGCTGG | 23 |
| forward | | |
| primer | | |
| PP | GAGTCGTAGGAGACAGAAGGTG | 24 |
| reverse | | |
| primer | | |
| SLC2A2 | ATGTCAGTGGGACTTGTGCTGC | 25 |
| forward | | |
| primer | | |
| SLC2A2 | AACTCAGCCACCATGAACCAGG | 26 |
| reverse | | |
| primer | | |
| GCK | CATCTCCGACTTCCTGGACAAG | 27 |
| forward | | |
| primer | | |
| GCK | TGGTCCAGTTGAGAAGGATGCC | 28 |
| reverse | | |
| primer | | |
| ABCC8 | GACGACAAGAGGACAGTGGTCT | 29 |
| forward | | |
| primer | | |
| ABCC8 | GCATTCAGACCTCTGGAAGTCC | 30 |
| reverse | | |
| primer | | |
| KCNJ11 | TGTGTCACCAGCATCCACTCCT | 31 |
| forward | | |
| primer | | |
| KCNJ11 | GTTCTGCACGATGAGGATCAGG | 32 |
| reverse | | |
| primer | | |
| IA2 | TGGAGATCCTGGCTGAGCATGT | 33 |
| forward | | |
| primer | | |
| IA2 | GGTCACATCAGCCAAAGACAGG | 34 |
| reverse | | |
| primer | | |
| KRT19 | AGCTAGAGGTGAAGATCCGCGA | 35 |
| forward | | |
| primer | | |
| KRT19 | GCAGGACAATCCTGGAGTTCTC | 36 |
| reverse | | |
| primer | | |
| CA2 | GTGACCTGGATTGTGCTCAAGG | 37 |
| forward | | |
| primer | | |
| CA2 | GTTGTCCACCATCAGTTCTTCGG | 38 |
| reverse | | |
| primer | | |
| HNF1B | CCCAGCAAATCTTGTACCAGGC | 39 |
| forward | | |
| primer | | |
| HNF1B | ACCTCAGTGACCAAGTTGGAGC | 40 |
| reverse | | |
| primer | | |
| SOX9 | AGGAAGCTCGCGGACCAGTAC | 41 |
| forward | | |
| primer | | |
| SOX9 | GGTGGTCCTTCTTGTGCTGCAC | 42 |
| reverse | | |
| primer | | |
| Human | AGGATAGGTTGTATTAGAAGAGGTTATTAAG | 43 |
| insulin | | |
| promoter | | |
| forward | | |
| primer | | |
| Human | CCCCTAAACTCACCCCCACATACTTC | 44 |
| insulin | | |
| promoter | | |
| reverse | | |
| primer | | |
| Sequence | CGGGGAGAGCATGGGGTTCGGAACGCCCTCCTCACCTGCGGCGAGGGCGCCC | 45 |
| flanking | ACCAGGAGCA | |
| the | | |
| gRNA | | |
| targeting | | |
| site of | | |
| wildtype | | |
| ALDH3B | | |
| 2. The | | |
| ALDH3B | | |
| 2 gRNA | | |
| targeting | | |
| site is | | |
| labelled | | |
| in bold | | |
| font. | | |
| Sequence | CGGGGAGAGCATGGGGTTCGGAACGCCCTCCTCACCTGCGAGGGCGCCCACC | 46 |
| of | AGGAGCA | |
| ALDH3B | | |
| 2 indel | | |
| mutant 1 | | |
| (Mut1). | | |
| The | | |
| ALDH3B | | |
| 2 gRNA | | |
| targeting | | |
| site is | | |
| labelled | | |
| in bold | | |
| font. | | |
| Sequence | CGGGGAGAGCATGGGGTTCGGAACGCCCTCCTCACCTGGCGGCGAGGGCGCC | 47 |
| of | CACCAGGAGCA | |
| ALDH3B | | |
| 2 indel | | |
| mutant 2 | | |
| (Mut). | | |
| Underlining | | |
| indicates | | |
| position | | |
| of an | | |
| inserted | | |
| nucleotide. | | |
| The | | |
| ALDH3B | | |
| 2 gRNA | | |
| targeting | | |
| site is | | |
| labelled | | |
| in bold | | |
| font. | | |
| Sequence | CGGGGAGAGCATGGGGTTCGGAACGCCCTCCTCACCTGCGCCCACCAGGAGC | 48 |
| of | A | |
| ALDH3B | | |
| 2 indel | | |
| mutant 3 | | |
| (Mut3). | | |
| The | | |
| ALDH3B | | |
| 2 gRNA | | |
| targeting | | |
| site is | | |
| labelled | | |
| in bold | | |
| font. | | |
| Sequence | CGGGGAGAGCATGGGGTTCGGAACGCAGGAGCA | 49 |
| of | | |
| ALDH3B | | |
| 2 indel | | |
| mutant 4 | | |
| (Mut4). | | |
| The | | |
| ALDH3B | | |
| 2 gRNA | | |
| targeting | | |
| site is | | |
| labelled | | |
| in bold | | |
| font. | | |
| Sequence | CGGGGAGAGCATGGGGTTCGGAACCCCACCAGGAGCA | 50 |
| of | | |
| ALDH3B | | |
| 2 indel | | |
| mutant 5 | | |
| (Mut5) | | |
| Sequence | CGGGGAGAGCATGGGGTTCGGAACGCCCTCCTCACCTGCGGCGCGAGGGCGC | 51 |
| of | CCACCAGGAGCA | |
| ALDH3B | | |
| 2 indel | | |
| mutant 6 | | |
| (Mut6). | | |
| Underlining | | |
| indicates | | |
| position | | |
| of an | | |
| inserted | | |
| nucleotide. | | |
| The | | |
| ALDH3B | | |
| 2 gRNA | | |
| targeting | | |
| site is | | |
| labelled | | |
| in bold | | |
| font. | | |
| Sequence | CGGGGAGAGCATGGGGTTCGGAACGCCCTCCTCACCTGCGGCGCCCACCAGG | 52 |
| of | AGCA | |
| ALDH3B | | |
| 2 indel | | |
| mutant 7 | | |
| (Mut7). | | |
| The | | |
| ALDH3B | | |
| 2 gRNA | | |
| targeting | | |
| site is | | |
| labelled | | |
| in bold | | |
| font. | | |
| Sequence | CGGGGAGAGCATGGGGTTCGGAACGCCCTCCTCACCTGCGGCGAGGAGCA | 53 |
| of | | |
| ALDH3B | | |
| 2 indel | | |
| mutant 8 | | |
| (Mut8). | | |
| The | | |
| ALDH3B | | |
| 2 gRNA | | |
| targeting | | |
| site is | | |
| labelled | | |
| in bold | | |
| font. | | |
| Sequence | CGGGGAGAGCATGGGGTTCGGAACGCCCTCCTCACCTGCCGGCGAGGGCGCC | 54 |
| of | CACCAGGAGCA | |
| ALDH3B | | |
| 2 indel | | |
| mutant 9 | | |
| (Mut9). | | |
| Underlining | | |
| indicates | | |
| position | | |
| of an | | |
| inserted | | |
| nucleotide. | | |
| The | | |
| ALDH3B | | |
| 2 gRNA | | |
| targeting | | |
| site is | | |
| labelled | | |
| in bold | | |
| font. | | |
| Sequence | CGGGGAGAGCATGGGGTTCGGAACGCCCTCCTCACCTGCGGAGGCGCCCACC | 55 |
| of | AGGAGCA | |
| ALDH3B | | |
| 2 indel | | |
| mutant | | |
| 10 | | |
| (Mut10). | | |
| The | | |
| ALDH3B | | |
| 2 gRNA | | |
| targeting | | |
| site is | | |
| labelled | | |
| in bold | | |
| font. | | |
| Sequence | CGGGGAGAGCATGGGGTTCGGAACGCCCTCCCTGAGGGCGCCCACCAGGAGC | 56 |
| of | A | |
| ALDH3B | | |
| 2 indel | | |
| mutant | | |
| 11 | | |
| (Mut11). | | |
| The | | |
| ALDH3B | | |
| 2 gRNA | | |
| targeting | | |
| site is | | |
| labelled | | |
| in bold | | |
| font. | | |
| Sequence | CGGGGAGAGCATGGGGTTCGGAACGCCCTCCTCACCTGCGGGCGCCCACCAG | 57 |
| of | GAGCA | |
| ALDH3B | | |
| 2 indel | | |
| mutant | | |
| 12 | | |
| (Mut12). | | |
| The | | |
| ALDH3B | | |
| 2 gRNA | | |
| targeting | | |
| site is | | |
| labelled | | |
| in bold | | |
| font. | | |
| Sequence | CGGGGAGAGCATGGGGTTCGGAACGCCCTCCTCACCAGGAGCA | 58 |
| of | | |
| ALDH3B | | |
| 2 indel | | |
| mutant | | |
| 13 | | |
| (Mut13). | | |
| The | | |
| ALDH3B | | |
| 2 gRNA | | |
| targeting | | |
| site is | | |
| labelled | | |
| in bold | | |
| font. | | |

### DESCRIPTION OF THE EMBODIMENTS

### I. Methods of Inducing Insulin Production or Transdifferentiating Pancreatic Duct Cells

In some embodiments, a method comprises inducing insulin production from pancreatic duct cells by inhibiting aldehyde dehydrogenase family 3 member B2 (ALDH3B2) protein or inhibiting the expression of ALDH3B2 gene. Such an increase in insulin production by pancreatic duct cells would allow for these cells to release insulin, either via baseline release or in response to glucose.

ALDH3B2 (also known as ALDH8) is an enzyme that in humans is encoded by the ALDH3B2 gene (Gene ID: 222). The ALDH3B2 gene encodes a member of the aldehyde dehydrogenase family, which are isozymes that play a major role in the detoxification of aldehydes generated by alcohol metabolism and lipid peroxidation.

In some embodiments, the insulin production is maintained after withdrawing the inhibiting of ALDH3B2. In other words, inhibiting ALDH3B2 protein or inhibiting the expression of ALDH3B2 gene may cause an irreversible change in pancreatic duct cells to produce insulin. In some embodiments, pancreatic duct cells may be irreversibly transdifferentiated into insulin-producing beta-like cells after inhibiting of ALDH3B2.

In some embodiments, inhibiting ALDH3B2 protein or inhibiting the expression of ALDH3B2 gene can transdifferentiate pancreatic duct cells into insulin-producing beta-like cells. By “transdifferentiate,” it is meant that pancreatic duct cells take on one or more characteristic generally associated with insulin-producing beta cells. As used herein, “beta-like cell” refers to a cell that secretes insulin in response to glucose. In other words, inhibiting ALDH3B2 protein or inhibiting the expression of ALDH3B2 gene can cause pancreatic duct cells to take on one or more characteristics associated with pancreatic beta cells, including being able to secrete insulin in response to glucose. This transdifferentiation of pancreatic duct cells into beta-like cells can allow for increased insulin release in a subject in response to glucose and help to treat diabetes as in methods described herein.

In some embodiments, a method of inducing insulin production from pancreatic duct cells in response to glucose comprises administering an agent that inhibits ALDH3B2 protein or an agent that inhibits the expression of ALDH3B2 gene.

In some embodiments, a method of inducing insulin production from pancreatic duct cells in response to glucose comprises administering an agent that inhibits ALDH3B2 protein or an agent that inhibits the expression of ALDH3B2 gene, wherein the insulin production in response to glucose is maintained after withdrawing the agent.

In some embodiments, a method of transdifferentiating pancreatic duct cells into insulin-producing beta-like cells comprises administering an agent that inhibits ALDH3B2 protein or an agent that inhibits the expression of ALDH3B2 gene.

In some embodiments, some or most of pancreatic duct cells in a sample are transdifferentiated into beta-like cells after inhibition of ALDH3B2. In some embodiments, the efficiency of transdifferentiating is 5% or more, 10% or more, 15% or more, 20% or more, or 25% or more. In some embodiments, the transdifferentiation may occur in vivo within a subject.

In some embodiments, a method of transdifferentiating pancreatic duct cells into insulin-producing beta-like cells comprises administering an agent that inhibits ALDH3B2 protein or an agent that inhibits the expression of ALDH3B2 gene, wherein the efficiency of transdifferentiating is 5% or more, 10% or more, 15% or more, 20% or more, or 25% or more.

In some embodiments, a method of transdifferentiating pancreatic duct cells into insulin-producing beta-like cells comprises administering an agent that inhibits ALDH3B2 protein or an agent that inhibits the expression of ALDH3B2 gene, wherein the insulin-producing beta-like cells are maintained after withdrawing the agent. In some embodiments, a method of transdifferentiating pancreatic duct cells into insulin-producing beta-like cells comprises administering an agent that inhibits ALDH3B2 protein or an agent that inhibits the expression of ALDH3B2 gene, wherein the efficiency of transdifferentiating is 5% or more, 10% or more, 15% or more, 20% or more, or 25% or more and wherein the insulin-producing beta-like cells are maintained after withdrawing the agent.

In some embodiments, the insulin-producing beta-like cells comprise insulin granules. In this way, the beta-like cells have a supply of insulin that is ready to be released, such as in response to glucose. In some embodiments, the beta-like cells may have regulated release of insulin in response to glucose uptake.

In some embodiments, a method of transdifferentiating pancreatic duct cells into insulin-producing beta-like cells comprises administering an agent that inhibits ALDH3B2 protein or an agent that inhibits the expression of ALDH3B2 gene, wherein the insulin-producing beta-like cells comprise insulin granules. In some embodiments, a method of transdifferentiating pancreatic duct cells into insulin-producing beta-like cells comprises administering an agent that inhibits ALDH3B2 protein or an agent that inhibits the expression of ALDH3B2 gene, wherein the efficiency of transdifferentiating is 5% or more, 10% or more, 15% or more, 20% or more, or 25% or more and wherein the insulin-producing beta-like cells comprise insulin granules. In some embodiments, a method of transdifferentiating pancreatic duct cells into insulin-producing beta-like cells comprises administering an agent that inhibits ALDH3B2 protein or an agent that inhibits the expression of ALDH3B2 gene, wherein the efficiency of transdifferentiating is 5% or more, 10% or more, 15% or more, 20% or more, or 25% or more, the insulin-producing beta-like cells are maintained after withdrawing the agent, and the insulin-producing beta-like cells comprise insulin granules.

In some embodiments, inhibiting ALDH3B2 protein or inhibiting the expression of ALDH3B2 gene produces epigenetic changes in pancreatic duct cells. As used herein, “an epigenetic change” refers to a change that does not alter a DNA sequence. For example, DNA methylation is an epigenetic change that works by adding a chemical group (methyl) to DNA. In the case of the insulin promoter, demethylation can impact on beta cell maturation and tissue-specific insulin gene expression (See, for example, Kuroda et al. PLoS ONE 4(9):e6953 (2009)). In some embodiments, the epigenetic change induced by inhibiting ALDH3B2 protein or inhibiting the expression of ALDH3B2 gene is a reduction in DNA methylation in the insulin gene locus. In some embodiments, a reduction in DNA methylation in the insulin gene locus leads to an increase in insulin production.

In some embodiments, a method of inducing insulin production from pancreatic duct cells in response to glucose comprises administering an agent that inhibits ALDH3B2 protein or an agent that inhibits the expression of ALDH3B2 gene, wherein administration of the agent results in epigenetic changes.

In some embodiments, a method of inducing insulin production from pancreatic duct cells in response to glucose comprises administering an agent that inhibits ALDH3B2 protein or an agent that inhibits the expression of ALDH3B2 gene, wherein the insulin production in response to glucose is maintained after withdrawing the agent, and wherein administration of the agent results in epigenetic changes.

In some embodiments, a method of transdifferentiating pancreatic duct cells into insulin-producing beta-like cells comprises administering an agent that inhibits ALDH3B2 protein or an agent that inhibits the expression of ALDH3B2 gene, wherein administration of the agent results in epigenetic changes. In some embodiments, a method of transdifferentiating pancreatic duct cells into insulin-producing beta-like cells comprises administering an agent that inhibits ALDH3B2 protein or an agent that inhibits the expression of ALDH3B2 gene, wherein the efficiency of transdifferentiating is 5% or more, 10% or more, 15% or more, 20% or more, or 25% or more and wherein administration of the agent results in epigenetic changes. In some embodiments, a method of transdifferentiating pancreatic duct cells into insulin-producing beta-like cells comprises administering an agent that inhibits ALDH3B2 protein or an agent that inhibits the expression of ALDH3B2 gene, wherein the efficiency of transdifferentiating is 5% or more, 10% or more, 15% or more, 20% or more, or 25% or more, the insulin-producing beta-like cells are maintained after withdrawing the agent, and administration of the agent results in epigenetic changes. In some embodiments, a method of transdifferentiating pancreatic duct cells into insulin-producing beta-like cells comprises administering an agent that inhibits ALDH3B2 protein or an agent that inhibits the expression of ALDH3B2 gene, wherein the efficiency of transdifferentiating is 5% or more, 10% or more, 15% or more, 20% or more, or 25% or more, the insulin-producing beta-like cells are maintained after withdrawing the agent, the insulin-producing beta-like cells comprise insulin granules, and administration of the agent results in epigenetic changes.

In some embodiments, a method of transdifferentiating pancreatic duct cells into insulin-producing beta-like cells comprises administering an agent that inhibits ALDH3B2 protein or an agent that inhibits the expression of ALDH3B2 gene, wherein administration of the agent results in epigenetic changes, wherein the epigenetic change is a reduction in DNA methylation in the insulin gene locus. In some embodiments, a method of transdifferentiating pancreatic duct cells into insulin-producing beta-like cells comprises administering an agent that inhibits ALDH3B2 protein or an agent that inhibits the expression of ALDH3B2 gene, wherein the efficiency of transdifferentiating is 5% or more, 10% or more, 15% or more, 20% or more, or 25% or more and wherein administration of the agent results in epigenetic changes, and wherein the epigenetic change is a reduction in DNA methylation in the insulin gene locus. In some embodiments, a method of transdifferentiating pancreatic duct cells into insulin-producing beta-like cells comprises administering an agent that inhibits ALDH3B2 protein or an agent that inhibits the expression of ALDH3B2 gene, wherein the efficiency of transdifferentiating is 5% or more, 10% or more, 15% or more, 20% or more, or 25% or more, the insulin-producing beta-like cells are maintained after withdrawing the agent, and administration of the agent results in epigenetic changes, and wherein the epigenetic change is a reduction in DNA methylation in the insulin gene locus. In some embodiments, a method of transdifferentiating pancreatic duct cells into insulin-producing beta-like cells comprises administering an agent that inhibits ALDH3B2 protein or an agent that inhibits the expression of ALDH3B2 gene, wherein the efficiency of transdifferentiating is 5% or more, 10% or more, 15% or more, 20% or more, or 25% or more, the insulin-producing beta-like cells are maintained after withdrawing the agent, the insulin-producing beta-like cells comprise insulin granules, and administration of the agent results in epigenetic changes, and wherein the epigenetic change is a reduction in DNA methylation in the insulin gene locus.

There are a number of means to inhibit ALDH3B2 activity. In some embodiments, an agent inhibits ALDH3B2 protein or an agent inhibits the expression of ALDH3B2 gene. Since ALDH3B2 is an enzyme, in some embodiments, an agent may inhibit ALDH3B2 activity without changing the expression level of ALDH3B2 protein. In some embodiments, expression of the ALDH3B2 gene is decreased, without an effect of the activity of individual ALDH3B2 protein molecule. In other words, ALDH3B2 may be inhibited by decreasing its activity, decreasing expression of ALDH3B2 gene, or both.

### A. Inhibitors of ALDH3B2 Protein Amount or Activity of the Protein

Different methods may be used to inhibit ALDH3B2 protein. Generally, inhibiting ALDH3B2 protein comprises any method that decreases the amount or activity of ALDH3B2 protein.

ALDH3B2 is an enzyme, and accordingly its enzymatic activity can be decreased without changing the amount of ALDH3B2 expressed. In some embodiments, inhibiting ALDH3B2 protein is performed by treatment with one or more inhibitor of the ALDH3B2 protein.

Different inhibitors of ALDH3B2 protein are known in the art (See, for example, Koppaka et al., Pharmacol Rev 64:520-539 (2012)). In some embodiments, the inhibitor of the ALDH3B2 protein is an ALDH inhibitor. In some embodiments, the inhibitor is a 4-amino-4-methyl-2-pentyne-1-al, benomyl, chloral hydrate, chlorpropamide analogs, citral, coprine, cyanamide, daidzin, 4-(diethylamino)benzaldehyde, disulfiram, gossypol, kynurenine metabolites, molinate, nitroglycerin, and/or pargyline. In some embodiments, inhibiting ALDH3B2 protein is performed with a single agent. In some embodiments, inhibiting ALDH3B2 protein is performed with multiple agents.

Further, assays are known in the art for determining whether a given agent is an inhibitor of ALDH3B2 protein. For example, an in vitro fluorescent assay measuring NADH production by ALDH3B2 protein has been described (see Kitamura et al., Biochem J465:79-87 (2015)). Any agent that inhibits ALDH3B2 protein in such an assay of enzymatic activity would be considered an inhibitor of ALDH3B2 protein. Similar assays may be developed with spectrophotometric analysis. An inhibition of 25% or greater, 50% or greater, 75% or greater, or 90% or greater in an enzymatic activity assay would indicate that a given agent is an inhibitor of ALDH3B2 protein.

In some embodiments, a method of inducing insulin production from pancreatic duct cells in response to glucose comprises administering an agent that inhibits ALDH3B2 protein or an agent that inhibits the expression of ALDH3B2 gene, wherein the agent is one or more inhibitor of the ALDH3B2 protein.

In some embodiments, a method of inducing insulin production from pancreatic duct cells in response to glucose comprises administering an agent that inhibits ALDH3B2 protein or an agent that inhibits the expression of ALDH3B2 gene, wherein the agent is one or more inhibitor of the ALDH3B2 protein, wherein the inhibitor is 4-amino-4-methyl-2-pentyne-1-al, benomyl, chloral hydrate, chlorpropamide analogs, citral, coprine, cyanamide, daidzin, 4-(diethylamino)benzaldehyde, disulfiram, gossypol, kynurenine metabolites, molinate, nitroglycerin, and/or pargyline.

In some embodiments, a method of treating a subject with diabetes comprises administering an agent that inhibits ALDH3B2 protein or an agent that inhibits the expression of ALDH3B2 gene, wherein the agent is one or more inhibitor of the ALDH3B2 protein.

In some embodiments, a method of treating a subject with diabetes comprises administering an agent that inhibits ALDH3B2 protein or an agent that inhibits the expression of ALDH3B2 gene, wherein the agent is one or more inhibitor of the ALDH3B2 protein, and wherein the inhibitor is 4-amino-4-methyl-2-pentyne-1-al, benomyl, chloral hydrate, chlorpropamide analogs, citral, coprine, cyanamide, daidzin, 4-(diethylamino)benzaldehyde, disulfiram, gossypol, kynurenine metabolites, molinate, nitroglycerin, and/or pargyline.

Also disclosed herein are methods of preventing the development of diabetes. In some embodiments, the development of type 1 diabetes is prevented. In some embodiments, the development of type 2 diabetes is prevented.

In some embodiments, a method of preventing the development of diabetes comprises screening a subject for risk factors for diabetes; determining if the subject has increased risk of developing diabetes; and administering an agent that inhibits ALDH3B2 protein or an agent that inhibits the expression of ALDH3B2 gene to a subject that has an increased risk of diabetes, wherein the agent is one or more inhibitor of the ALDH3B2 protein.

In some embodiments, a method of preventing the development of diabetes comprises screening a subject for risk factors for diabetes; determining if the subject has increased risk of developing diabetes; and administering an agent that inhibits ALDH3B2 protein or an agent that inhibits the expression of ALDH3B2 gene to a subject that has an increased risk of diabetes, wherein the agent is one or more inhibitor of the ALDH3B2 protein, and wherein the inhibitor is 4-amino-4-methyl-2-pentyne-1-al, benomyl, chloral hydrate, chlorpropamide analogs, citral, coprine, cyanamide, daidzin, 4-(diethylamino)benzaldehyde, disulfiram, gossypol, kynurenine metabolites, molinate, nitroglycerin, and/or pargyline.

### B. Inhibition of ALDH3B2 Gene Expression

In some embodiments, methods are performed by inhibiting expression of ALDH3B2 gene. As used herein, inhibiting expression of ALDH3B2 gene may mean decreasing or eliminating its expression.

In some embodiments, inhibiting expression of ALDH3B2 is performed by gene editing. As used herein, “gene editing” refers to any alteration of the genetic material of a living organism by inserting, replacing, or deleting a DNA sequence. In some embodiments, the gene editing is by the CRISPR/Cas system, transcription activator-like effector nucleases (TALENs), homing endonuclease, or zinc-finger nucleases (ZFNs). In some embodiments, the components of the gene editing system are delivered by virus transduction of pancreatic duct cells. In some embodiments, the components of the gene editing system are delivered by a lipid nanoparticle (e.g., LNP, Lipoplex, or similar).

In some embodiments, inhibiting expression of ALDH3B2 is performed using a small interfering RNA (siRNA). In some embodiments, the siRNA is a small hairpin RNA (shRNA).

In some embodiments, a method of inducing insulin production from pancreatic duct cells in response to glucose comprises administering an agent that inhibits ALDH3B2 protein or an agent that inhibits the expression of ALDH3B2 gene, wherein the agent decreases or eliminates expression of the ALDH3B2 protein.

In some embodiments, a method of inducing insulin production from pancreatic duct cells in response to glucose comprises administering an agent that inhibits ALDH3B2 protein or an agent that inhibits the expression of ALDH3B2 gene, wherein the agent decreases or eliminates expression of the ALDH3B2 protein, wherein the agent is a gene editing tool.

In some embodiments, a method of inducing insulin production from pancreatic duct cells in response to glucose comprises administering an agent that inhibits ALDH3B2 protein or an agent that inhibits the expression of ALDH3B2 gene, wherein the agent decreases or eliminates expression of the ALDH3B2 protein, wherein the agent is a gene editing tool, and wherein the gene editing tool is a CRISPR/Cas system, transcription activator-like effector nuclease (TALEN), homing endonuclease, or zinc-finger nuclease (ZFN).

In some embodiments, a method of inducing insulin production from pancreatic duct cells in response to glucose comprises administering an agent that inhibits ALDH3B2 protein or an agent that inhibits the expression of ALDH3B2 gene, wherein the agent decreases or eliminates expression of the ALDH3B2 protein, wherein the agent is a gene editing tool, and wherein the gene editing tool is a CRISPR/Cas system, transcription activator-like effector nuclease (TALEN), homing endonuclease, or zinc-finger nuclease (ZFN), wherein the gene editing tool is delivered by virus transduction or lipid nanoparticle delivery.

In some embodiments, a method of transdifferentiating pancreatic duct cells into insulin-producing beta-like cells comprises administering an agent that inhibits ALDH3B2 protein or an agent that inhibits the expression of ALDH3B2 gene, wherein the agent decreases or eliminates expression of the ALDH3B2 protein.

In some embodiments, a method of transdifferentiating pancreatic duct cells into insulin-producing beta-like cells comprises administering an agent that inhibits ALDH3B2 protein or an agent that inhibits the expression of ALDH3B2 gene, wherein the agent decreases or eliminates expression of the ALDH3B2 protein, wherein the agent is a gene editing tool.

In some embodiments, a method of transdifferentiating pancreatic duct cells into insulin-producing beta-like cells comprises administering an agent that inhibits ALDH3B2 protein or an agent that inhibits the expression of ALDH3B2 gene, wherein the agent decreases or eliminates expression of the ALDH3B2 protein, wherein the agent is a gene editing tool, and wherein the gene editing tool is a CRISPR/Cas system, transcription activator-like effector nuclease (TALEN), homing endonuclease, or zinc-finger nuclease (ZFN).

In some embodiments, a method of transdifferentiating pancreatic duct cells into insulin-producing beta-like cells comprises administering an agent that inhibits ALDH3B2 protein or an agent that inhibits the expression of ALDH3B2 gene, wherein the agent decreases or eliminates expression of the ALDH3B2 protein, wherein the agent is a gene editing tool, and wherein the gene editing tool is a CRISPR/Cas system, transcription activator-like effector nuclease (TALEN), homing endonuclease, or zinc-finger nuclease (ZFN), wherein the gene editing tool is delivered by virus transduction or lipid nanoparticle delivery.

In some embodiments, a method of treating a subject with diabetes comprises administering an agent that inhibits ALDH3B2 protein or an agent that inhibits the expression of ALDH3B2 gene, wherein the agent decreases or eliminates expression of the ALDH3B2 protein.

In some embodiments, a method of treating a subject with diabetes comprises administering an agent that inhibits ALDH3B2 protein or an agent that inhibits the expression of ALDH3B2 gene, wherein the agent decreases or eliminates expression of the ALDH3B2 protein, wherein the agent is a gene editing tool.

In some embodiments, a method of treating a subject with diabetes comprises administering an agent that inhibits ALDH3B2 protein or an agent that inhibits the expression of ALDH3B2 gene, wherein the agent decreases or eliminates expression of the ALDH3B2 protein, wherein the agent is a gene editing tool, and wherein the gene editing tool is a CRISPR/Cas system, transcription activator-like effector nuclease (TALEN), homing endonuclease, or zinc-finger nuclease (ZFN).

In some embodiments, a method of treating a subject with diabetes comprises administering an agent that inhibits ALDH3B2 protein or an agent that inhibits the expression of ALDH3B2 gene, wherein the agent decreases or eliminates expression of the ALDH3B2 protein, wherein the agent is a gene editing tool, and wherein the gene editing tool is a CRISPR/Cas system, transcription activator-like effector nuclease (TALEN), homing endonuclease, or zinc-finger nuclease (ZFN), wherein the gene editing tool is delivered by virus transduction or lipid nanoparticle delivery.

In some embodiments, a method of preventing the development of diabetes comprises screening a subject for risk factors for diabetes; determining if the subject has increased risk of developing diabetes; and administering an agent that inhibits ALDH3B2 protein or an agent that inhibits the expression of ALDH3B2 gene to a subject that has an increased risk of diabetes, wherein the agent decreases or eliminates expression of the ALDH3B2 protein.

In some embodiments, a method of preventing the development of diabetes comprises screening a subject for risk factors for diabetes; determining if the subject has increased risk of developing diabetes; and administering an agent that inhibits ALDH3B2 protein or an agent that inhibits the expression of ALDH3B2 gene to a subject that has an increased risk of diabetes, wherein the agent decreases or eliminates expression of the ALDH3B2 protein, wherein the agent is a gene editing tool.

In some embodiments, a method of preventing the development of diabetes comprises screening a subject for risk factors for diabetes; determining if the subject has increased risk of developing diabetes; and administering an agent that inhibits ALDH3B2 protein or an agent that inhibits the expression of ALDH3B2 gene to a subject that has an increased risk of diabetes, wherein the agent decreases or eliminates expression of the ALDH3B2 protein, wherein the agent is a gene editing tool, and wherein the gene editing tool is a CRISPR/Cas system, transcription activator-like effector nuclease (TALEN), homing endonuclease, or zinc-finger nuclease (ZFN).

In some embodiments, a method of treating a subject with diabetes comprises administering an agent that inhibits ALDH3B2 protein or an agent that inhibits the expression of ALDH3B2 gene, wherein the agent decreases or eliminates expression of the ALDH3B2 protein, wherein the agent is a gene editing tool, and wherein the gene editing tool is a CRISPR/Cas system, transcription activator-like effector nuclease (TALEN), homing endonuclease, or zinc-finger nuclease (ZFN), wherein the gene editing tool is delivered by virus transduction or lipid nanoparticle delivery.

In some embodiments, a method of inducing insulin production from pancreatic duct cells in response to glucose comprises administering an agent that inhibits ALDH3B2 protein or an agent that inhibits the expression of ALDH3B2 gene, wherein the agent decreases or eliminates expression of the ALDH3B2 protein, wherein the agent is a small interfering RNA.

In some embodiments, a method of transdifferentiating pancreatic duct cells into insulin-producing beta-like cells comprises administering an agent that inhibits ALDH3B2 protein or an agent that inhibits the expression of ALDH3B2 gene, wherein the agent decreases or eliminates expression of the ALDH3B2 protein, wherein the agent is a small interfering RNA.

In some embodiments, a method of treating a subject with diabetes comprises administering an agent that inhibits ALDH3B2 protein or an agent that inhibits the expression of ALDH3B2 gene, wherein the agent decreases or eliminates expression of the ALDH3B2 protein, wherein the agent is a small interfering RNA.

In some embodiments, a method of preventing the development of diabetes comprises screening a subject for risk factors for diabetes; determining if the subject has increased risk of developing diabetes; and administering an agent that inhibits ALDH3B2 protein or an agent that inhibits the expression of ALDH3B2 gene to a subject that has an increased risk of diabetes, wherein the agent decreases or eliminates expression of the ALDH3B2 protein, wherein the agent is a small interfering RNA.

### C. Additional Treatments

In some embodiments, inhibiting ALDH3B2 protein or inhibiting the expression of ALDH3B2 gene is performed in combination with an additional treatment. In some embodiments, the additional treatment is one or more agent that can treat diabetes. In some embodiments, inhibiting ALDH3B2 protein or inhibiting the expression of ALDH3B2 gene has an additive or synergistic effect together with another agent in improving treatment of diabetes. In some embodiments, 4-amino-4-methyl-2-pentyne-1-al, benomyl, chloral hydrate, chlorpropamide analogs, citral, coprine, cyanamide, daidzin, 4-(diethylamino)benzaldehyde, disulfiram, gossypol, kynurenine metabolites, molinate, nitroglycerin, and/or pargyline.

In some embodiments, an agent that inhibits ALDH3B2 protein or an agent that inhibits the expression of ALDH3B2 gene is administered in combination with an additional treatment. In some embodiments, the agent is administered in combination with an additional treatment, wherein the additional treatment is a glucagon-like peptide analog or agonist, dipeptidyl peptidase-4 inhibitor, amylin analog, biguanide, thiazolidinedione, sulfonylurea, meglitinide, alpha-glucosidase inhibitor, or sodium/glucose transporter 2 inhibitor.

### II. Methods of Treatment and Prevention

Glucose levels in the blood are normally tightly regulated to maintain an appropriate source of energy for cells of the body. Insulin and glucagon are principal hormones that regulate blood glucose levels. In response to an increase in blood glucose, such as after a meal, insulin is released from beta cells of the pancreas. Insulin regulates the metabolism of carbohydrates and fats by promoting uptake of glucose from the blood into fat and skeletal muscle. Insulin also promotes fat storage and inhibits the release of glucose by the liver. Regulation of insulin levels is a primary means for the body to regulate glucose in the blood.

When glucose levels in the blood are decreased, insulin is no longer released and instead glucagon is released from the alpha cells of the pancreas. Glucagon causes the liver to convert stored glycogen into glucose and to release this glucose into the bloodstream. Thus, insulin and glucagon work in concert to regulate blood glucose levels.

In one embodiment, treatment of diabetes mellitus is to administer an inhibitor of ALDH3B2 to a subject to lower blood glucose. In some embodiments, a method of treating a subject with diabetes comprises administering an agent that inhibits ALDH3B2 protein or inhibits the expression of ALDH3B2 gene, wherein the administering increases insulin secretion from the pancreas. In some embodiments, this insulin secretion is in response to glucose. In some embodiments, the insulin secretion is from pancreatic duct cells or insulin-producing beta-like cells transdifferentiated from pancreatic duct cells.

In some embodiments, a method of treating a subject with diabetes comprises administering an agent that inhibits ALDH3B2 protein or an agent that inhibits the expression of ALDH3B2 gene, wherein the agent increases insulin secretion from the pancreas.

In some embodiments, a method of treating a subject with diabetes comprises administering an agent that inhibits ALDH3B2 protein or an agent that inhibits the expression of ALDH3B2 gene, wherein the agent increases insulin secretion from the pancreas, and wherein the insulin secretion is from pancreatic duct cells or insulin-producing beta-like cells transdifferentiated from pancreatic duct cells.

In some embodiments, the subject has elevated blood sugar levels as compared to a normal subject. In some embodiments, the subject has a blood sugar level higher than 11.1 mmol/liter or 200 mg/dl.

In some embodiments, a method of treating a subject with diabetes comprises administering an agent that inhibits ALDH3B2 protein or an agent that inhibits the expression of ALDH3B2 gene, wherein the subject has a blood sugar level higher than 11.1 mmol/liter or 200 mg/dl. In some embodiments, a method of treating a subject with diabetes comprises administering an agent that inhibits ALDH3B2 protein or an agent that inhibits the expression of ALDH3B2 gene, wherein the agent increases insulin secretion from the pancreas, and wherein the subject has a blood sugar level higher than 11.1 mmol/liter or 200 mg/dl. In some embodiments, a method of treating a subject with diabetes comprises administering an agent that inhibits ALDH3B2 protein or an agent that inhibits the expression of ALDH3B2 gene, wherein the agent increases insulin secretion from the pancreas, wherein the insulin secretion is from pancreatic duct cells or insulin-producing beta-like cells transdifferentiated from pancreatic duct cells, and wherein the subject has a blood sugar level higher than 11.1 mmol/liter or 200 mg/dl.

Hyperglycemia refers to an increased level of glucose in the blood. Hyperglycemia can be associated with high levels of sugar in the urine, frequent urination, and increased thirst. Diabetes mellitus refers to a medical state of hyperglycemia.

The American Diabetes Association (ADA) suggests that fasting plasma glucose (FPG) levels of 100 mg/dL to 125 mg/dL or HbA1c levels of 5.7% to 6.4% may be considered hyperglycemia and may indicate that a subject is at high risk of developing diabetes mellitus (i.e. prediabetes, see ADA Guidelines 2015).

The ADA states that a diagnosis of diabetes mellitus may be made in a number of ways. A diagnosis of diabetes mellitus can be made in a subject displaying an HbA1c level of ≥6.5%, an FPG levels of ≥126 mg/dL, a 2-hour plasma glucose of ≥200 mg/dL during an OGTT, or a random plasma glucose level≥200 mg/dL in a subject with classic symptoms of hyperglycemia. In some embodiments, the subject has a blood sugar level higher than 11.1 mmol/liter or 200 mg/dl.

Diabetes mellitus can be broken into type 1 and type 2. Type 1 diabetes mellitus (previously known as insulin-dependent diabetes or juvenile diabetes) is an autoimmune disease characterized by destruction of the insulin-producing beta cells of the pancreas. Classic symptoms of type 1 diabetes mellitus are frequent urination, increased thirst, increased hunger, and weight loss. Subjects with type 1 diabetes mellitus are dependent on administration of insulin for survival.

In some embodiments, the subject has type 2 diabetes and/or insulin resistance. In some embodiments, the subject retains pancreatic beta cells, but the subject does not have sufficient insulin release in response to glucose.

In the absence of regulation of glucose levels in subjects with diabetes, a range of serious complications may be seen. These include atherosclerosis, kidney disease, stroke, nerve damage, and blindness.

In some embodiments, the subject treated has diabetes mellitus based on diagnosis criteria of the American Diabetes Association. In some embodiments, the subject with diabetes mellitus has an HbA1c level of ≥6.5%. In some embodiments, the subject with diabetes mellitus has an FPG levels of ≥126 mg/dL. In some embodiments, the subject with diabetes mellitus has a 2-hour plasma glucose of ≥200 mg/dL during an OGTT. In some embodiments, the subject with diabetes mellitus has a random plasma glucose level≥200 mg/dL or 11.1 mmol/L. In some embodiments, the subject with diabetes mellitus has a random plasma glucose level≥200 mg/dL or 11.1 mmol/L with classic symptoms of hyperglycemia.

In some embodiments, a method of treating a subject with diabetes comprises administering an agent that inhibits ALDH3B2 protein or an agent that inhibits the expression of ALDH3B2 gene, wherein subject has autoimmune diabetes. In some embodiments, a method of treating a subject with diabetes comprises administering an agent that inhibits ALDH3B2 protein or an agent that inhibits the expression of ALDH3B2 gene, wherein the agent increases insulin secretion from the pancreas, and wherein subject has autoimmune diabetes. In some embodiments, a method of treating a subject with diabetes comprises administering an agent that inhibits ALDH3B2 protein or an agent that inhibits the expression of ALDH3B2 gene, wherein the agent increases insulin secretion from the pancreas, wherein the insulin secretion is from pancreatic duct cells or insulin-producing beta-like cells transdifferentiated from pancreatic duct cells, and wherein the subject has autoimmune diabetes. In some embodiments, a method of treating a subject with diabetes comprises administering an agent that inhibits ALDH3B2 protein or an agent that inhibits the expression of ALDH3B2 gene, wherein the agent increases insulin secretion from the pancreas, wherein the insulin secretion is from pancreatic duct cells or insulin-producing beta-like cells transdifferentiated from pancreatic duct cells, wherein the subject has a blood sugar level higher than 11.1 mmol/liter or 200 mg/dl, and wherein the subject has autoimmune diabetes.

In some embodiments, a method of treating a subject with diabetes comprises administering an agent that inhibits ALDH3B2 protein or an agent that inhibits the expression of ALDH3B2 gene, wherein subject has autoimmune diabetes, wherein the subject has type 1 diabetes or autoimmune diabetes induced by an immunotherapy. In some embodiments, a method of treating a subject with diabetes comprises administering an agent that inhibits ALDH3B2 protein or an agent that inhibits the expression of ALDH3B2 gene, wherein the agent increases insulin secretion from the pancreas, and wherein subject has autoimmune diabetes, wherein the subject has type 1 diabetes or autoimmune diabetes induced by an immunotherapy. In some embodiments, a method of treating a subject with diabetes comprises administering an agent that inhibits ALDH3B2 protein or an agent that inhibits the expression of ALDH3B2 gene, wherein the agent increases insulin secretion from the pancreas, wherein the insulin secretion is from pancreatic duct cells or insulin-producing beta-like cells transdifferentiated from pancreatic duct cells, and wherein the subject has autoimmune diabetes, wherein the subject has type 1 diabetes or autoimmune diabetes induced by an immunotherapy. In some embodiments, a method of treating a subject with diabetes comprises administering an agent that inhibits ALDH3B2 protein or an agent that inhibits the expression of ALDH3B2 gene, wherein the agent increases insulin secretion from the pancreas, wherein the insulin secretion is from pancreatic duct cells or insulin-producing beta-like cells transdifferentiated from pancreatic duct cells, wherein the subject has a blood sugar level higher than 11.1 mmol/liter or 200 mg/dl, and wherein the subject has autoimmune diabetes, wherein the subject has type 1 diabetes or autoimmune diabetes induced by an immunotherapy.

In some embodiments, a method of treating a subject with diabetes comprises administering an agent that inhibits ALDH3B2 protein or an agent that inhibits the expression of ALDH3B2 gene, wherein subject has type 2 diabetes and/or insulin resistance. In some embodiments, a method of treating a subject with diabetes comprises administering an agent that inhibits ALDH3B2 protein or an agent that inhibits the expression of ALDH3B2 gene, wherein the agent increases insulin secretion from the pancreas, and wherein subject has type 2 diabetes and/or insulin resistance. In some embodiments, a method of treating a subject with diabetes comprises administering an agent that inhibits ALDH3B2 protein or an agent that inhibits the expression of ALDH3B2 gene, wherein the agent increases insulin secretion from the pancreas, wherein the insulin secretion is from pancreatic duct cells or insulin-producing beta-like cells transdifferentiated from pancreatic duct cells, and wherein the subject has type 2 diabetes and/or insulin resistance. In some embodiments, a method of treating a subject with diabetes comprises administering an agent that inhibits ALDH3B2 protein or an agent that inhibits the expression of ALDH3B2 gene, wherein the agent increases insulin secretion from the pancreas, wherein the insulin secretion is from pancreatic duct cells or insulin-producing beta-like cells transdifferentiated from pancreatic duct cells, wherein the subject has a blood sugar level higher than 11.1 mmol/liter or 200 mg/dl, and wherein the subject has type 2 diabetes and/or insulin resistance.

In some embodiments, a method of treating a subject with diabetes comprises administering an agent that inhibits ALDH3B2 protein or an agent that inhibits the expression of ALDH3B2 gene, wherein the subject has lower baseline blood glucose after administration. In some embodiments, a method of treating a subject with diabetes comprises administering an agent that inhibits ALDH3B2 protein or an agent that inhibits the expression of ALDH3B2 gene, wherein the agent increases insulin secretion from the pancreas, and wherein the subject has lower baseline blood glucose after administration. In some embodiments, a method of treating a subject with diabetes comprises administering an agent that inhibits ALDH3B2 protein or an agent that inhibits the expression of ALDH3B2 gene, wherein the agent increases insulin secretion from the pancreas, wherein the insulin secretion is from pancreatic duct cells or insulin-producing beta-like cells transdifferentiated from pancreatic duct cells, and wherein the subject has lower baseline blood glucose after administration. In some embodiments, a method of treating a subject with diabetes comprises administering an agent that inhibits ALDH3B2 protein or an agent that inhibits the expression of ALDH3B2 gene, wherein the agent increases insulin secretion from the pancreas, wherein the insulin secretion is from pancreatic duct cells or insulin-producing beta-like cells transdifferentiated from pancreatic duct cells, wherein the subject has a blood sugar level higher than 11.1 mmol/liter or 200 mg/dl, and wherein the subject has lower baseline blood glucose after administration. In some embodiments, a method of treating a subject with diabetes comprises administering an agent that inhibits ALDH3B2 protein or an agent that inhibits the expression of ALDH3B2 gene, wherein the agent increases insulin secretion from the pancreas, wherein the insulin secretion is from pancreatic duct cells or insulin-producing beta-like cells transdifferentiated from pancreatic duct cells, wherein the subject has a blood sugar level higher than 11.1 mmol/liter or 200 mg/dl, wherein the subject has autoimmune diabetes, and wherein the subject has lower baseline blood glucose after administration. In some embodiments, a method of treating a subject with diabetes comprises administering an agent that inhibits ALDH3B2 protein or an agent that inhibits the expression of ALDH3B2 gene, wherein the agent increases insulin secretion from the pancreas, wherein the insulin secretion is from pancreatic duct cells or insulin-producing beta-like cells transdifferentiated from pancreatic duct cells, wherein the subject has a blood sugar level higher than 11.1 mmol/liter or 200 mg/dl, wherein the subject has autoimmune diabetes, wherein the subject has type 1 diabetes or autoimmune diabetes induced by an immunotherapy, and wherein the subject has lower baseline blood glucose after administration.

In some embodiments, a method of treating a subject with diabetes comprises administering an agent that inhibits ALDH3B2 protein or an agent that inhibits the expression of ALDH3B2 gene, wherein the subject has lower blood glucose in response to a glucose challenge after administration. In some embodiments, a method of treating a subject with diabetes comprises administering an agent that inhibits ALDH3B2 protein or an agent that inhibits the expression of ALDH3B2 gene, wherein the agent increases insulin secretion from the pancreas, and wherein the subject has lower blood glucose in response to a glucose challenge after administration. In some embodiments, a method of treating a subject with diabetes comprises administering an agent that inhibits ALDH3B2 protein or an agent that inhibits the expression of ALDH3B2 gene, wherein the agent increases insulin secretion from the pancreas, wherein the insulin secretion is from pancreatic duct cells or insulin-producing beta-like cells transdifferentiated from pancreatic duct cells, and wherein the subject has lower blood glucose in response to a glucose challenge after administration. In some embodiments, a method of treating a subject with diabetes comprises administering an agent that inhibits ALDH3B2 protein or an agent that inhibits the expression of ALDH3B2 gene, wherein the agent increases insulin secretion from the pancreas, wherein the insulin secretion is from pancreatic duct cells or insulin-producing beta-like cells transdifferentiated from pancreatic duct cells, wherein the subject has a blood sugar level higher than 11.1 mmol/liter or 200 mg/dl, and wherein the subject has lower blood glucose in response to a glucose challenge after administration. In some embodiments, a method of treating a subject with diabetes comprises administering an agent that inhibits ALDH3B2 protein or an agent that inhibits the expression of ALDH3B2 gene, wherein the agent increases insulin secretion from the pancreas, wherein the insulin secretion is from pancreatic duct cells or insulin-producing beta-like cells transdifferentiated from pancreatic duct cells, wherein the subject has a blood sugar level higher than 11.1 mmol/liter or 200 mg/dl, wherein the subject has autoimmune diabetes, and wherein the subject has lower blood glucose in response to a glucose challenge after administration. In some embodiments, a method of treating a subject with diabetes comprises administering an agent that inhibits ALDH3B2 protein or an agent that inhibits the expression of ALDH3B2 gene, wherein the agent increases insulin secretion from the pancreas, wherein the insulin secretion is from pancreatic duct cells or insulin-producing beta-like cells transdifferentiated from pancreatic duct cells, wherein the subject has a blood sugar level higher than 11.1 mmol/liter or 200 mg/dl, wherein the subject has autoimmune diabetes, wherein the subject has type 1 diabetes or autoimmune diabetes induced by an immunotherapy, and wherein the subject has lower blood glucose in response to a glucose challenge after administration. In some embodiments, a method of treating a subject with diabetes comprises administering an agent that inhibits ALDH3B2 protein or an agent that inhibits the expression of ALDH3B2 gene, wherein the agent increases insulin secretion from the pancreas, wherein the insulin secretion is from pancreatic duct cells or insulin-producing beta-like cells transdifferentiated from pancreatic duct cells, wherein the subject has a blood sugar level higher than 11.1 mmol/liter or 200 mg/dl, wherein the subject has autoimmune diabetes, wherein the subject has type 1 diabetes or autoimmune diabetes induced by an immunotherapy, and wherein the subject has lower baseline blood glucose after administration and lower blood glucose in response to a glucose challenge after administration.

In some embodiments, a method of treating a subject with diabetes comprises administering an agent that inhibits ALDH3B2 protein or an agent that inhibits the expression of ALDH3B2 gene, wherein the subject has lower baseline blood glucose after administration. In some embodiments, a method of treating a subject with diabetes comprises administering an agent that inhibits ALDH3B2 protein or an agent that inhibits the expression of ALDH3B2 gene, wherein the agent increases insulin secretion from the pancreas, and wherein the subject has lower baseline blood glucose after administration. In some embodiments, a method of treating a subject with diabetes comprises administering an agent that inhibits ALDH3B2 protein or an agent that inhibits the expression of ALDH3B2 gene, wherein the agent increases insulin secretion from the pancreas, wherein the insulin secretion is from pancreatic duct cells or insulin-producing beta-like cells transdifferentiated from pancreatic duct cells, and wherein the subject has lower baseline blood glucose after administration. In some embodiments, a method of treating a subject with diabetes comprises administering an agent that inhibits ALDH3B2 protein or an agent that inhibits the expression of ALDH3B2 gene, wherein the agent increases insulin secretion from the pancreas, wherein the insulin secretion is from pancreatic duct cells or insulin-producing beta-like cells transdifferentiated from pancreatic duct cells, wherein the subject has a blood sugar level higher than 11.1 mmol/liter or 200 mg/dl, and wherein the subject has lower baseline blood glucose after administration. In some embodiments, a method of treating a subject with diabetes comprises administering an agent that inhibits ALDH3B2 protein or an agent that inhibits the expression of ALDH3B2 gene, wherein the agent increases insulin secretion from the pancreas, wherein the insulin secretion is from pancreatic duct cells or insulin-producing beta-like cells transdifferentiated from pancreatic duct cells, wherein the subject has a blood sugar level higher than 11.1 mmol/liter or 200 mg/dl, wherein the subject has type 2 diabetes and/or insulin resistance, and wherein the subject has lower baseline blood glucose after administration.

In some embodiments, a method of treating a subject with diabetes comprises administering an agent that inhibits ALDH3B2 protein or an agent that inhibits the expression of ALDH3B2 gene, wherein the subject has lower baseline blood glucose after administration, wherein the subject has lower blood glucose in response to a glucose challenge after administration. In some embodiments, a method of treating a subject with diabetes comprises administering an agent that inhibits ALDH3B2 protein or an agent that inhibits the expression of ALDH3B2 gene, wherein the agent increases insulin secretion from the pancreas, and wherein the subject has lower blood glucose in response to a glucose challenge after administration. In some embodiments, a method of treating a subject with diabetes comprises administering an agent that inhibits ALDH3B2 protein or an agent that inhibits the expression of ALDH3B2 gene, wherein the agent increases insulin secretion from the pancreas, wherein the insulin secretion is from pancreatic duct cells or insulin-producing beta-like cells transdifferentiated from pancreatic duct cells, and wherein the subject has lower blood glucose in response to a glucose challenge after administration. In some embodiments, a method of treating a subject with diabetes comprises administering an agent that inhibits ALDH3B2 protein or an agent that inhibits the expression of ALDH3B2 gene, wherein the agent increases insulin secretion from the pancreas, wherein the insulin secretion is from pancreatic duct cells or insulin-producing beta-like cells transdifferentiated from pancreatic duct cells, wherein the subject has a blood sugar level higher than 11.1 mmol/liter or 200 mg/dl, and wherein the subject has lower blood glucose in response to a glucose challenge after administration. In some embodiments, a method of treating a subject with diabetes comprises administering an agent that inhibits ALDH3B2 protein or an agent that inhibits the expression of ALDH3B2 gene, wherein the agent increases insulin secretion from the pancreas, wherein the insulin secretion is from pancreatic duct cells or insulin-producing beta-like cells transdifferentiated from pancreatic duct cells, wherein the subject has a blood sugar level higher than 11.1 mmol/liter or 200 mg/dl, wherein the subject has type 2 diabetes and/or insulin resistance, and wherein the subject has lower blood glucose in response to a glucose challenge after administration. In some embodiments, a method of treating a subject with diabetes comprises administering an agent that inhibits ALDH3B2 protein or an agent that inhibits the expression of ALDH3B2 gene, wherein the agent increases insulin secretion from the pancreas, wherein the insulin secretion is from pancreatic duct cells or insulin-producing beta-like cells transdifferentiated from pancreatic duct cells, wherein the subject has a blood sugar level higher than 11.1 mmol/liter or 200 mg/dl, wherein the subject has type 2 diabetes and/or insulin resistance, and wherein the subject has lower baseline blood glucose after administration and lower blood glucose in response to a glucose challenge after administration.

In some embodiments of the invention, the subject is a mammal. In some embodiments, the mammal is a human, non-human primate, cow, horse, pig, sheep, goat, dog, cat, or rodent. In some embodiments, the subject is a human subject. In some embodiments of the invention, the subject is a mammal, wherein the mammal is human.

In some embodiments, the subject has autoimmune diabetes. In some embodiments, the subject treated has type 1 diabetes mellitus.

In some embodiments, the subject treated has a relative decrease in insulin levels. In some embodiments, the subject treated has decreased beta cell mass. In some embodiments, the decrease in beta cell mass in a subject is due to an autoimmune disease.

Treatment of patients with therapeutics targeted to increase the body's immune response to cancers, termed immunotherapies, has also been associated with the development of autoimmune diabetes (See Alrifai T et al., Case Reports in Oncological Medicine 2019: Article ID 8781347). For example, immune checkpoint antibodies have been reported to cause immune-mediated damage of islet cells leading to induction of autoimmune diabetes similar to type 1 diabetes.

In some embodiments, the subject has autoimmune diabetes induced by an immunotherapy. In some embodiments, the immunotherapy is a checkpoint antibody. In some embodiments, the checkpoint antibody is an anti-PD-1 antibody, anti-PD-L1 antibody, or anti-CTLA-4 antibody.

In one embodiment, the method comprises lowering blood glucose levels in the diabetic subject to below about 200 mg/dL, 150 mg/dL, 100 mg/dL, or about 125 mg/dL.

In some embodiments, the subject has lower baseline blood glucose after the treating. In some embodiments, the subject has lower blood glucose in response to a glucose challenge after the treating. In some embodiments, the subject has lower baseline blood glucose and lower blood glucose in response to a glucose challenge after the treating.

Also encompassed is a method of preventing the development of diabetes by administering an agent that inhibits ALDH3B2 protein or an agent that inhibits the expression of ALDH3B2 gene to a subject that has an increased risk of diabetes. Certain individuals can be predicted to have a high risk for developing diabetes based on one or more risk factors, such as family history.

In some embodiments, a method of preventing the development of diabetes comprises screening a subject for risk factors for diabetes; determining if the subject has increased risk of developing diabetes; and administering an agent that inhibits ALDH3B2 protein or an agent that inhibits the expression of ALDH3B2 gene to a subject that has an increased risk of diabetes.

In some embodiments, screening a subject for risk factors comprises obtaining data on a genetic risk score that is based on the known diabetes-associated gene variants, a family history of diabetes, the presence of one or more autoantibodies against beta cell antigens that are known to predict disease risk, and/or abnormal glucose tolerance.

In some embodiments, a method of preventing the development of diabetes comprises screening a subject for risk factors for diabetes; determining if the subject has increased risk of developing diabetes; and administering an agent that inhibits ALDH3B2 protein or an agent that inhibits the expression of ALDH3B2 gene to a subject that has an increased risk of diabetes, wherein screening a subject for risk factors comprises obtaining data on a genetic risk score that is based on the known diabetes-associated gene variants, a family history of diabetes, the presence of one or more autoantibodies against beta cell antigens that are known to predict disease risk, and/or abnormal glucose tolerance

Many individuals with diabetes have a genetic susceptibility because their genome comprises one or more diabetes-associated gene variant. The presence of one or more of these variants leads to an increased risk of diabetes. As these diabetes-associated gene variants can be inherited, a subject with a positive family history of diabetes may have an increased risk of developing the disease.

A wide variety of diabetes-associated gene variants have been described (See, for example, Watkins R A et al., Transl Res. 164(2):110-21 (2014)). In some embodiments, the one or more diabetes-associated gene variant are comprised in one or more HLA gene. In some embodiments, the one or more diabetes-associated gene variant are HLA polymorphisms conferring greater risk for diabetes. In some embodiments, the one or more diabetes-associated gene variant are comprised in one or more non-HLA gene.

In some embodiments, a family history of diabetes is determined by patient history or a questionnaire. In some embodiments, a family history of diabetes is based on one or more sibling, parent, or grandparent having diabetes. In some embodiments, the family history is a family history of type 1 diabetes.

In some embodiments, autoantibody levels against beta cell antigens are measured to determine increased risk of developing type 1 diabetes. A wide variety of autoantibodies against beta cell antigens have described in the literature (See, for example, Watkins 2014). Autoantibody panels are commercially available to identify individuals at risk of developing type 1 diabetes. Inclusion of certain antibodies, such as anti-ZnT8, in autoantibody levels can predict individuals at risk of developing type 1 diabetes. In some embodiments, the presence of one or more autoantibodies is used to determine an increased risk of developing type 1 diabetes. In some embodiments, the number of autoantibodies or the titer of a specific autoantibody is used to determine an increased risk of developing type 1 diabetes.

In some embodiments, an abnormal glucose tolerance is used to determine an increased risk of developing diabetes. In some embodiments, a subject with increased risk of developing diabetes shows abnormal glucose tolerance results without presently meeting criteria for diabetes.

In some embodiments, a subject is determined to have an increased risk of developing diabetes based on the presence of more than one risk factor. For example, a subject with a positive family history for diabetes may be determined to also have an abnormal glucose tolerance. Multiple risk factors for diabetes can be assessed to determine a subject's risk of developing diabetes. In some embodiments, a subject's risk of developing diabetes is determined using an algorithm based on multiple risk factors (See, for example, Watkins 2014).

In some embodiments, a subject having an increased risk of diabetes is administered an agent that inhibits ALDH3B2 protein or an agent that inhibits the expression of ALDH3B2 gene. In some embodiments, administration of an agent that inhibits ALDH3B2 protein or an agent that inhibits the expression of ALDH3B2 gene prevents the development of diabetes in a subject with increased risk. In some embodiments, administration of an agent that inhibits ALDH3B2 protein or an agent that inhibits the expression of ALDH3B2 gene slows the time period until development of diabetes in a subject with increased risk.

### EXAMPLES

### Example 1. CRISPR Screen to Identify Regulators of Human Beta Cell Neogenesis

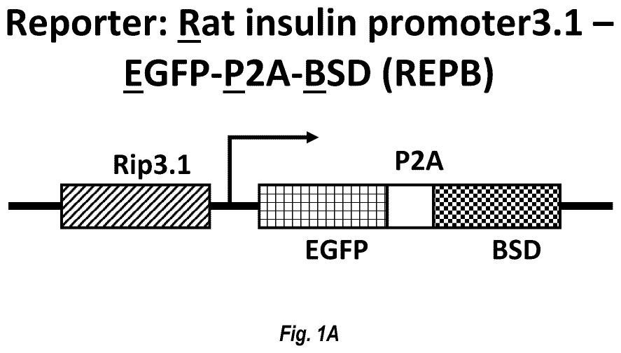

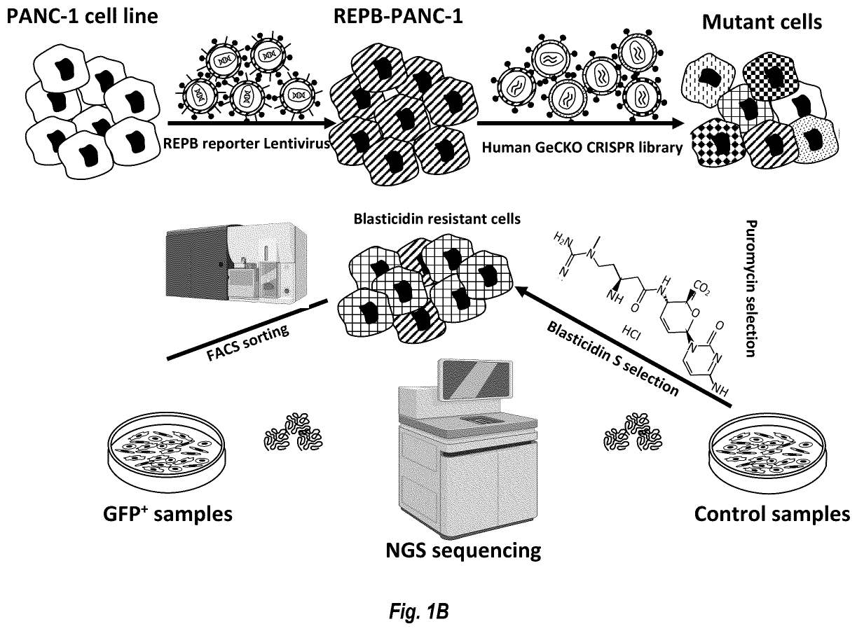

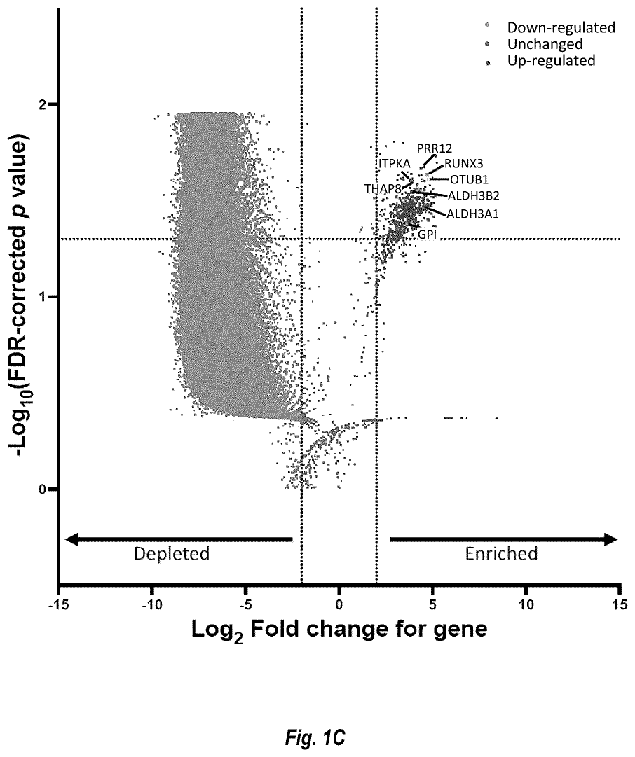

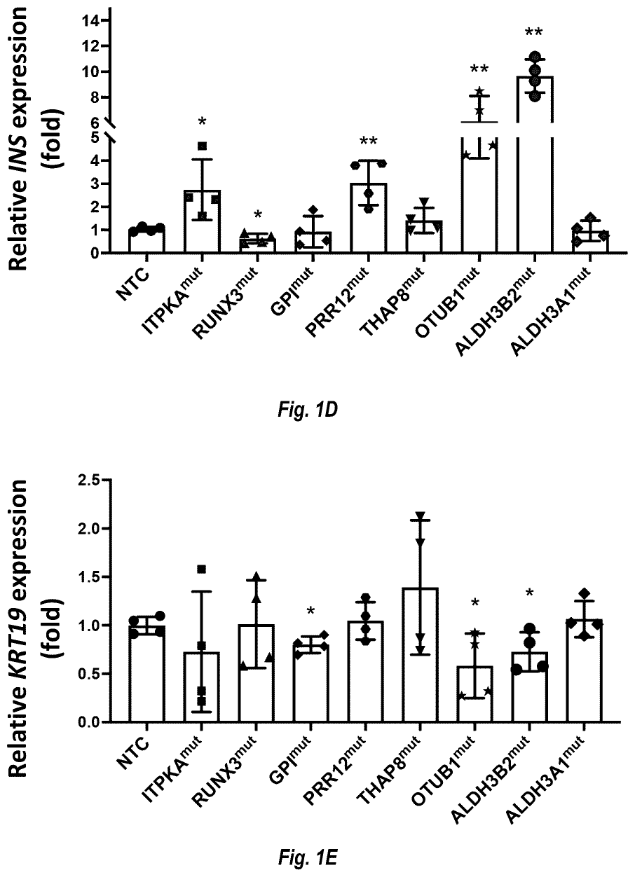

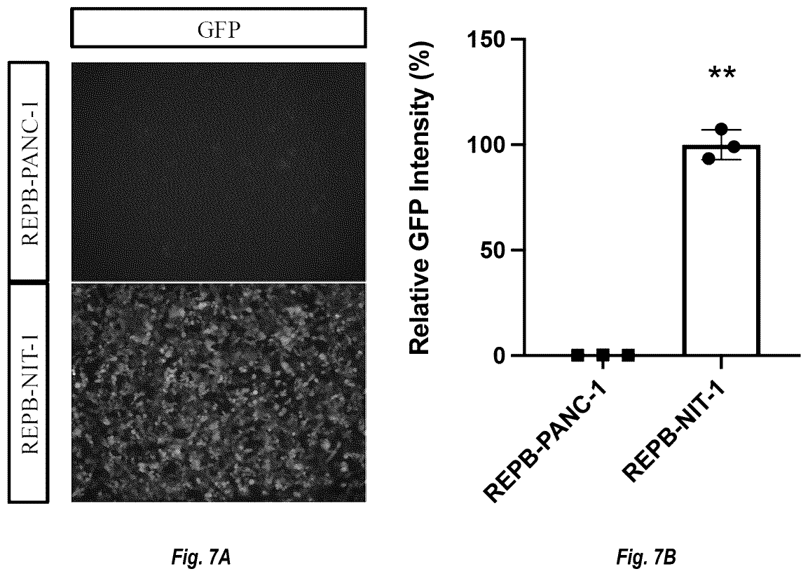

A screening strategy was designed to search for gene mutations that can transdifferentiate human pancreatic duct cells into beta cells. To ensure efficient gene editing and sufficient cell numbers for the genome-wide screen, an immortalized pancreatic cell line, the PANC-1 human pancreatic carcinoma cell line of ductal origin that maintains many of the differentiated characteristics of normal mammalian pancreatic ductal epithelium, was employed^{15, 16}. A reporter construct, rat insulin promoter (RIP)-EGFP-P2A-BSD (REPB), was introduced into the PANC-1 cells by lentiviral transduction (REPB-PANC-1) (FIG. 1A). The EGFP and Blasticidin-S deaminase (BSD) reporter genes are fused together with P2A peptide, where the EGFP reporter will enable visual and quantitative monitoring of insulin promoter activation, and the expression of BSD reporter gene confers resistance to blasticidin treatment, making it easy to select insulin promoter activated cells in our CRISPR screen. To validate the REPB reporter construct, the PANC-1 cells or a mouse beta cell line (NIT-1) were transduced with the REPB reporter lentivirus. As shown in FIGS. 7A and 7B, the EGFP expression can only be observed in the transduced NIT-1 cells (a mouse beta cell line), but not in PANC-1 cells. The REPB transduced PANC-1 cells, but not the NIT-1 cells, are sensitive to blasticidin treatment (data not shown).

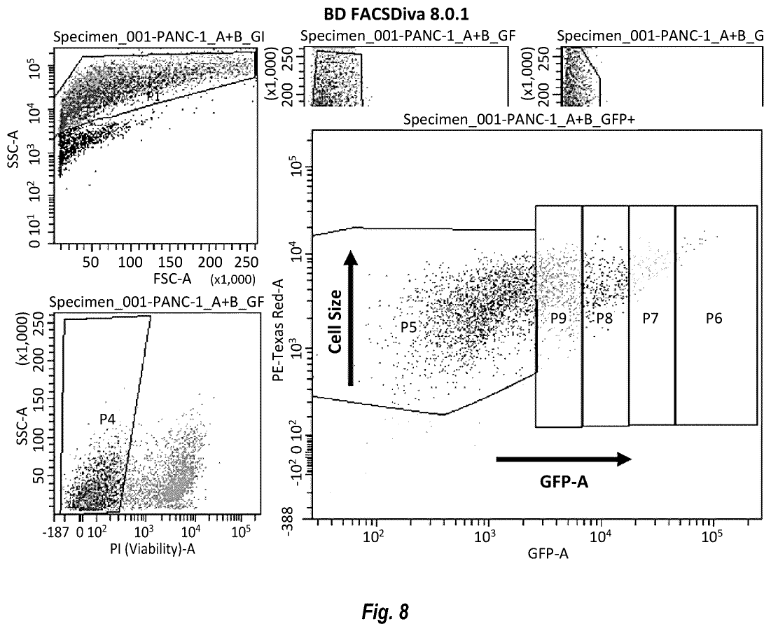

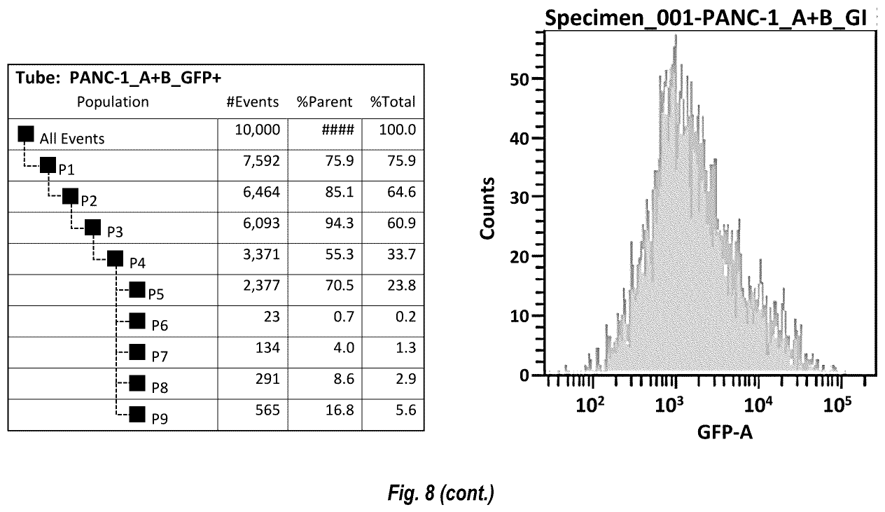

As illustrated in FIG. 1B, the REPB-PANC-1 cells were transduced with the human lentiviral genome-wide CRISPR knockout library (GeCKO v2)¹⁷ that comprises approximately 120,000 guide RNAs (gRNAs) targeting a total of 19,050 genes. A low multiplicity of infection (MOI) ˜0.3 was used to ensure most of the cells only carry one single mutation. Approximately 108 lentiviral library transduced REPB-PANC-1 cells were treated first with low dose of blasticidin (10 μg/ml) for 7 days, and then the blasticidin resistant cells were subjected to FACS sorting based on their EGFP intensity (FIG. 8). After next generation sequencing (NGS), the gRNA profile of the highest EGFP-expressing cells was compared to that of the cells without blasticidin selection and FACS sorting. As shown in FIG. 1C, most gRNAs were depleted from the EGFP high, blasticidin-resistant REPB-PANC-1 cells, whereas only few of them are significantly enriched.

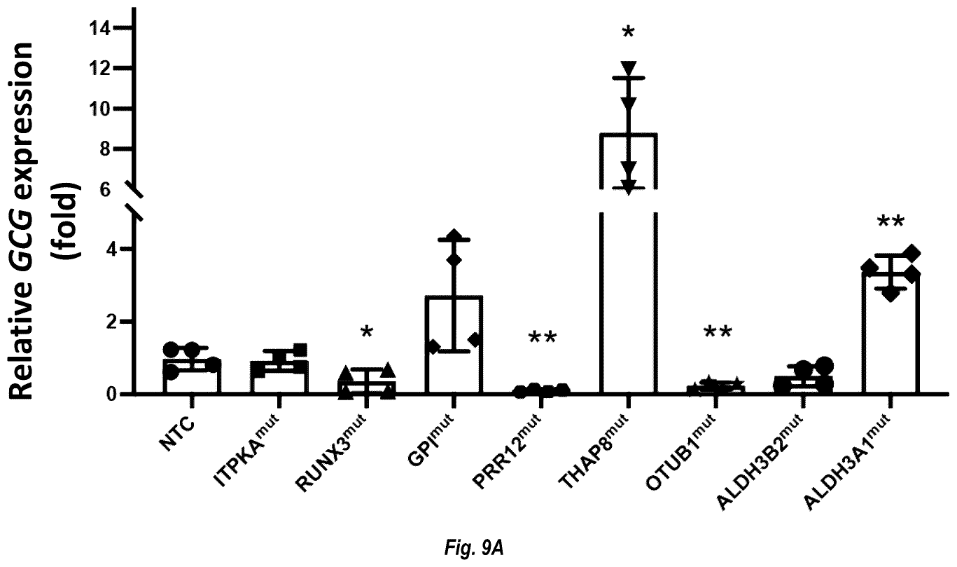

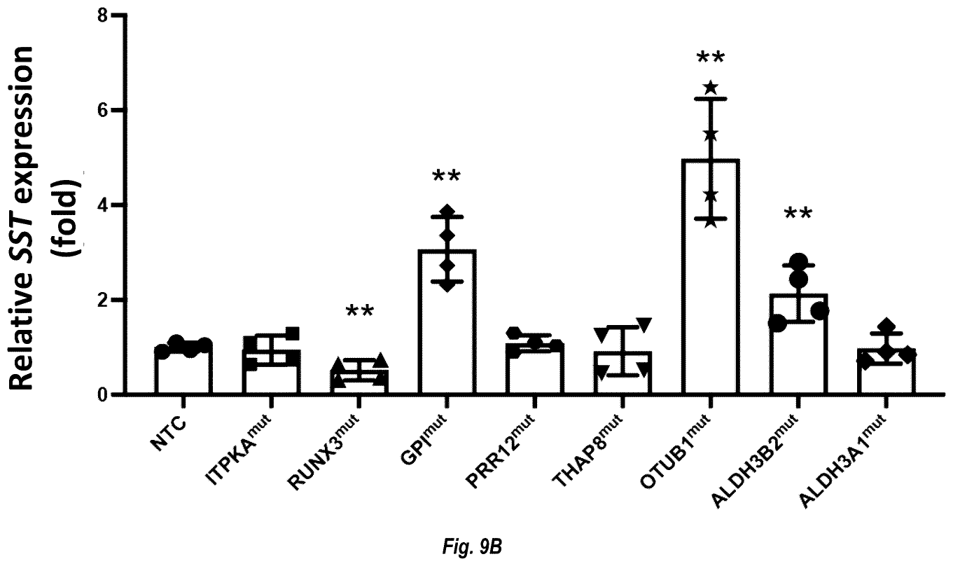

To validate the hits from the genome-wide CRISPR screen, 9 top candidate gRNAs were selected based the EGFP intensity and statistical significance, and generated individual mutant PANC-1 cell lines using the corresponding gRNAs that are identified from the CRISPR screen. Quantitative PCR (qPCR) was used to analyze the expression of endocrine marker genes including insulin (INS), glucagon (GCG) and somatostatin (SST), as well as pancreatic duct cell marker gene keratin 19 (KRT19). Indeed, several individual mutant PANC-1 cell lines showed differential expression of the examined marker genes (FIG. 1D, 1E and FIGS. 9A and 9B). In particular, the mutant PANC-1 cell line of one of the candidate genes, ALDH3B2, showed the highest INS expression and the lowest KRT19 compared to the non-targeting control (NTC) gRNA transduced PANC-1 cells.

ALDH3B2, also known as ALDH8, is one of the 19 human aldehyde dehydrogenase (ALDH) superfamily that convert various types of aldehydes to carboxylic acids 18. It is well-documented that ALDH genes are important regulators of stem cells and cell fate determination¹⁹. Another close member in the ALDH family, ALDH1A3, has recently been shown to contribute to pancreatic beta cell failure and de-differentiation²⁰ in type 2 diabetes. In addition, ALDH3B2 as an enzyme could potentially be an attractive therapeutic target using small molecules. Therefore, ALDH3B2 was prioritized for further in-depth validation and characterization.

### Example 2. Transdifferentiation of PANC-1 Cells into Insulin-Producing Beta-Like Cells

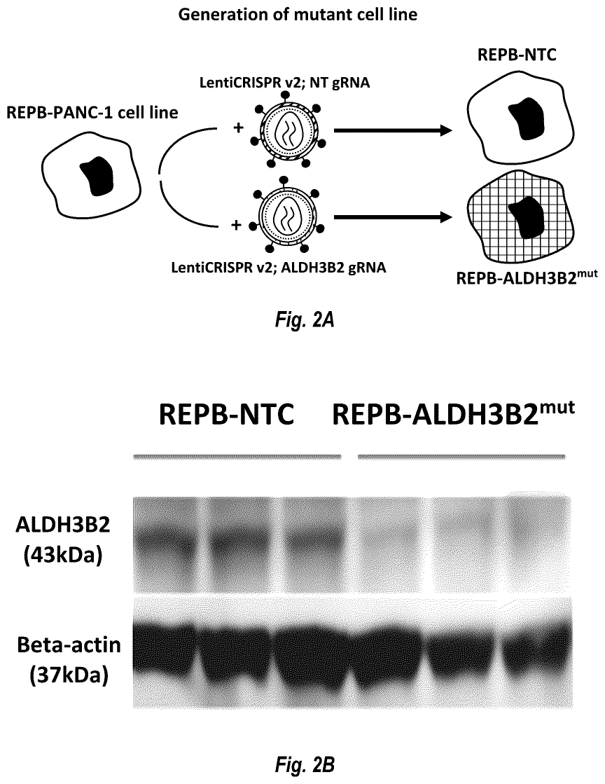

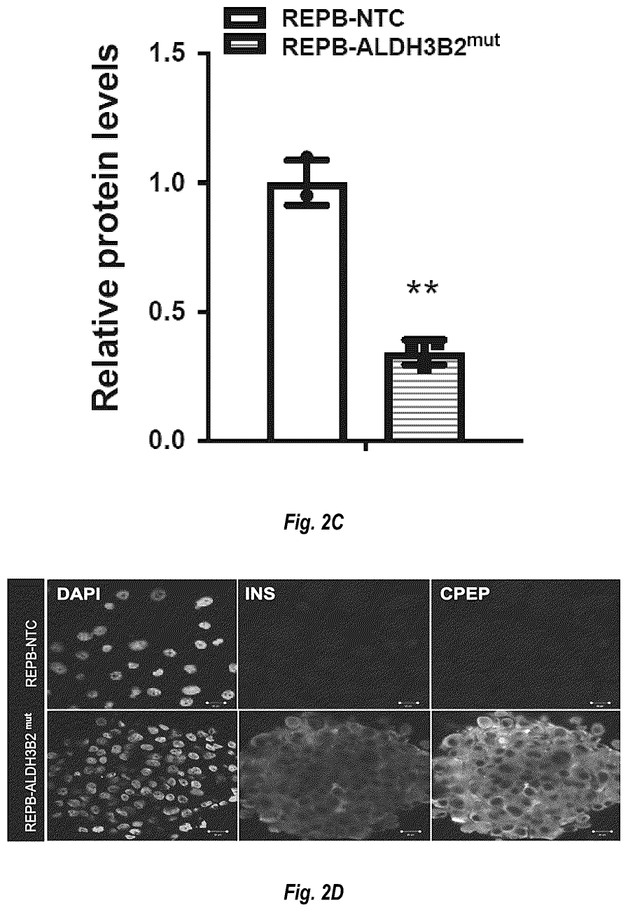

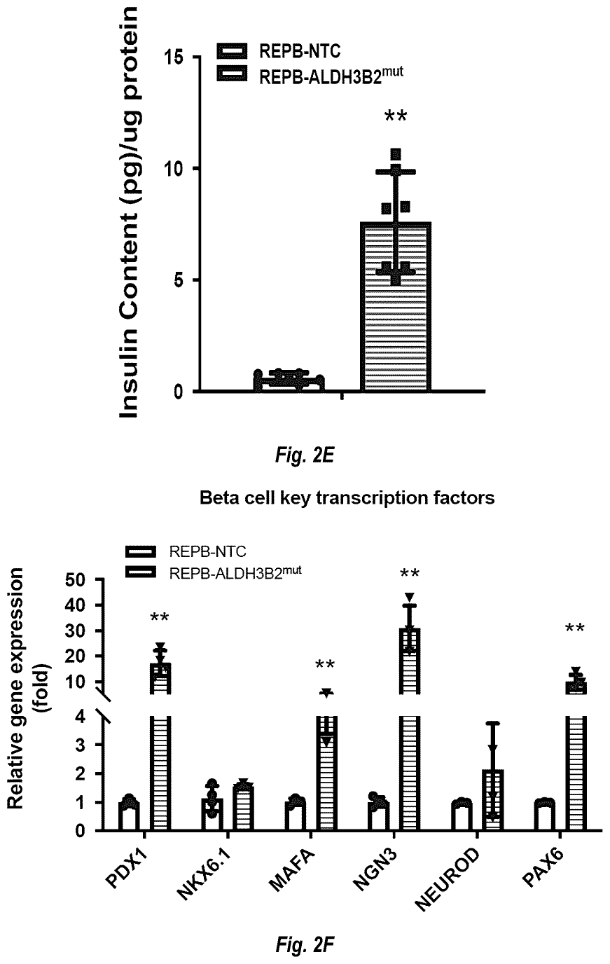

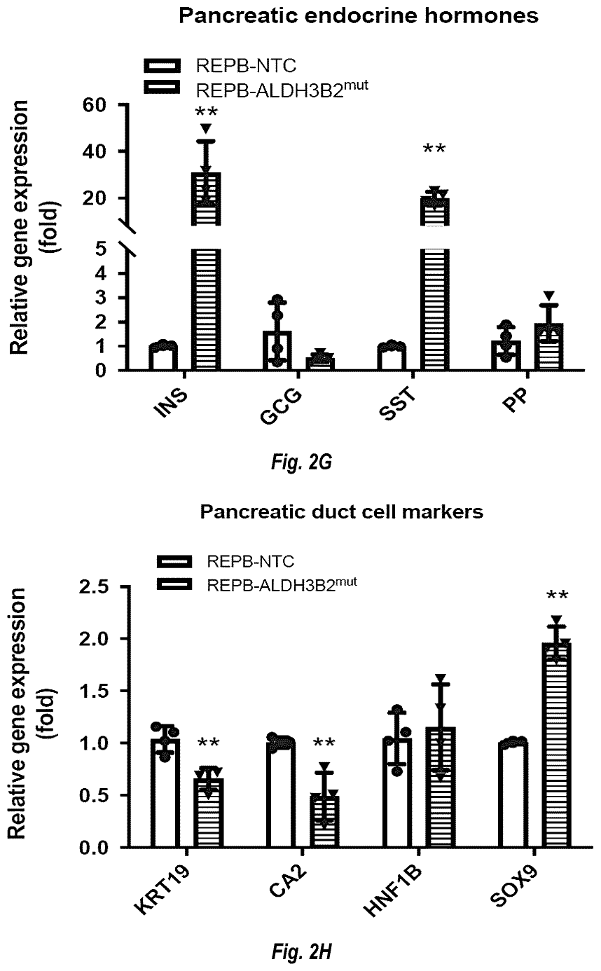

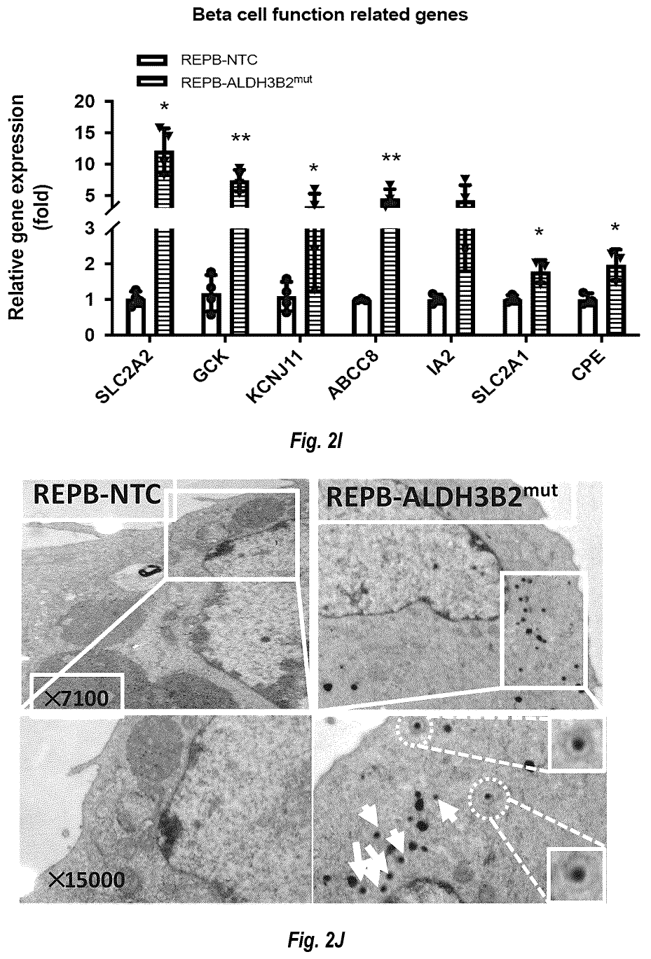

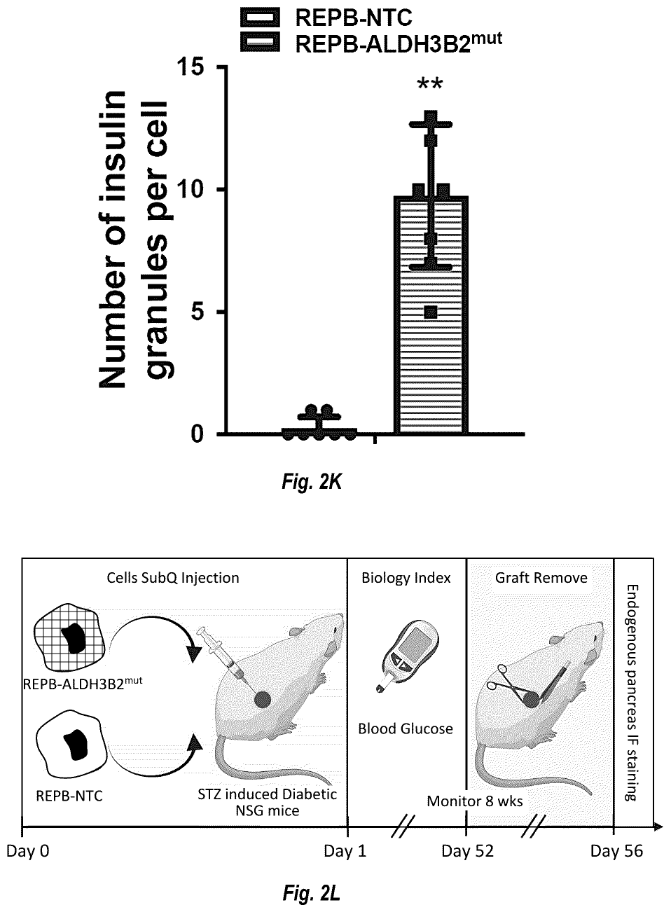

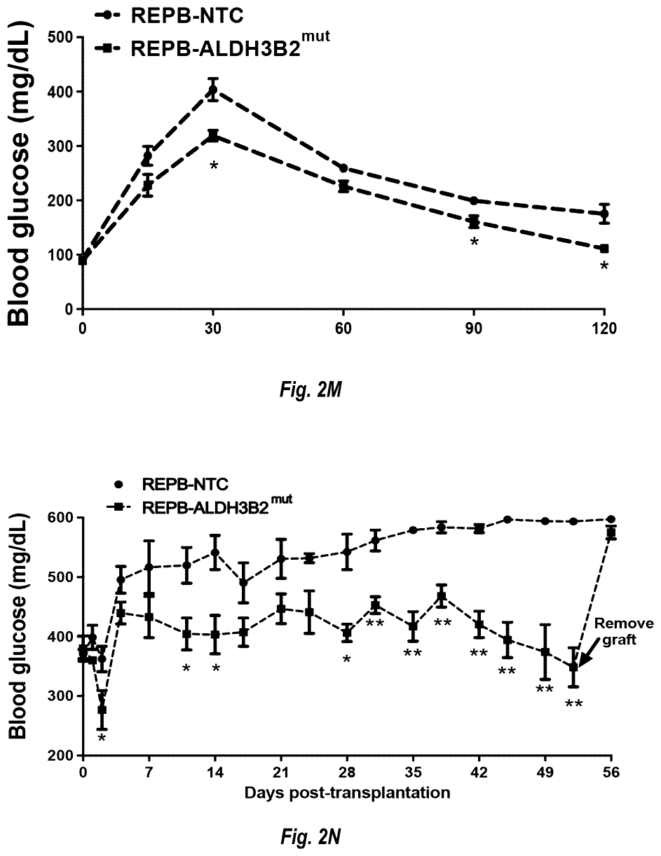

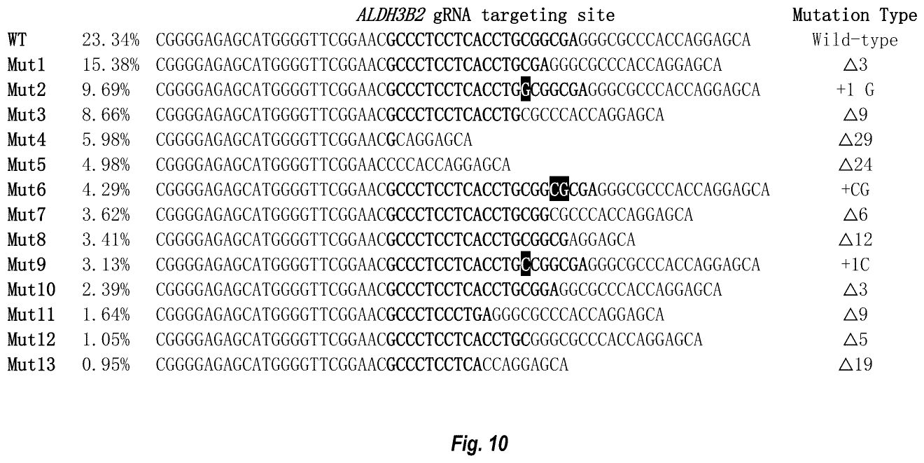

An ALDH3B2 mutant PANC-1 cell line was generated by lentiviral transduction of SpCas9 and ALDH3B2 gRNA into PANC-1 cells (FIG. 2A). Genome sequencing of the ALDH3B2mut PANC-1 cells showed over 75% of indel mutation in the ALDH3B2 locus (FIG. 10). The expression of ALDH3B2 in the mutant PANC-1 cells is significantly reduced (˜70%), as shown by western blot (FIGS. 2B and 2C). By immunofluorescence imaging, clusters of ALDH3B2 mutant PANC-1 cells were observed expressing human insulin and c-peptide, where neither insulin nor C-peptide can be detected in the non-targeting control (NTC) PANC-1 cells (FIG. 2D). The insulin content of the ALDH3B2 mutant PANC-1 was significantly increased compared to NTC-PANC-1 cells (FIG. 2E).

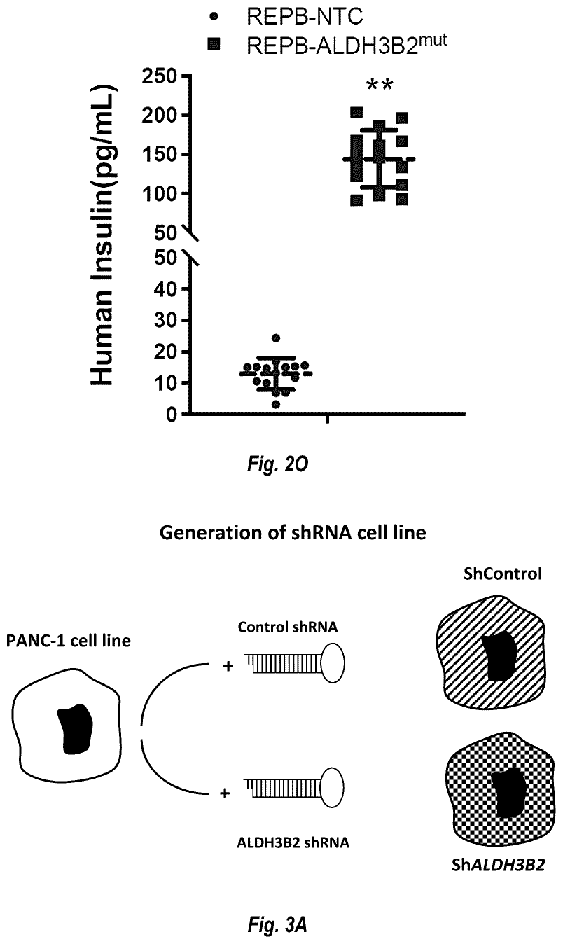

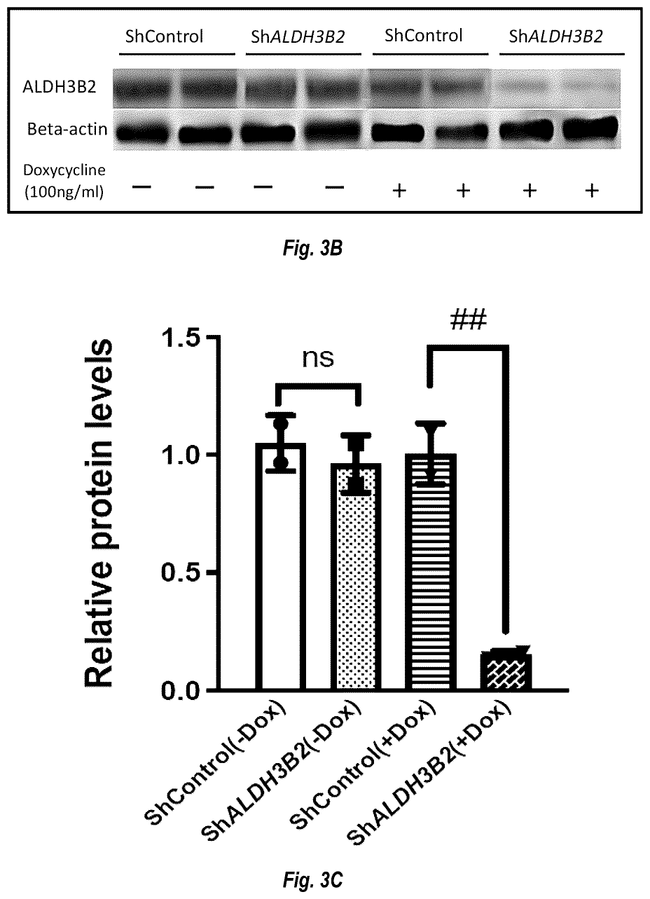

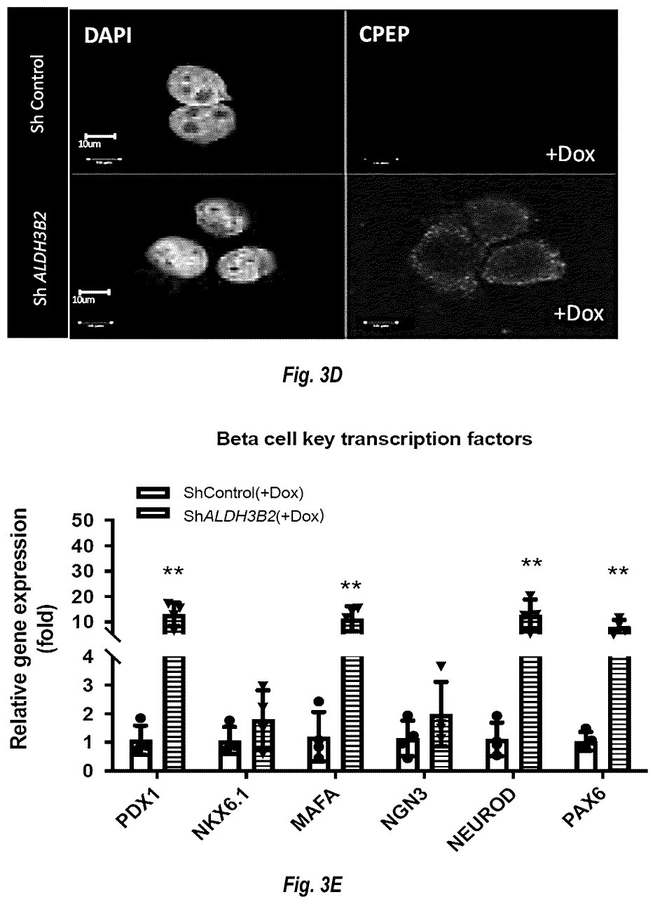

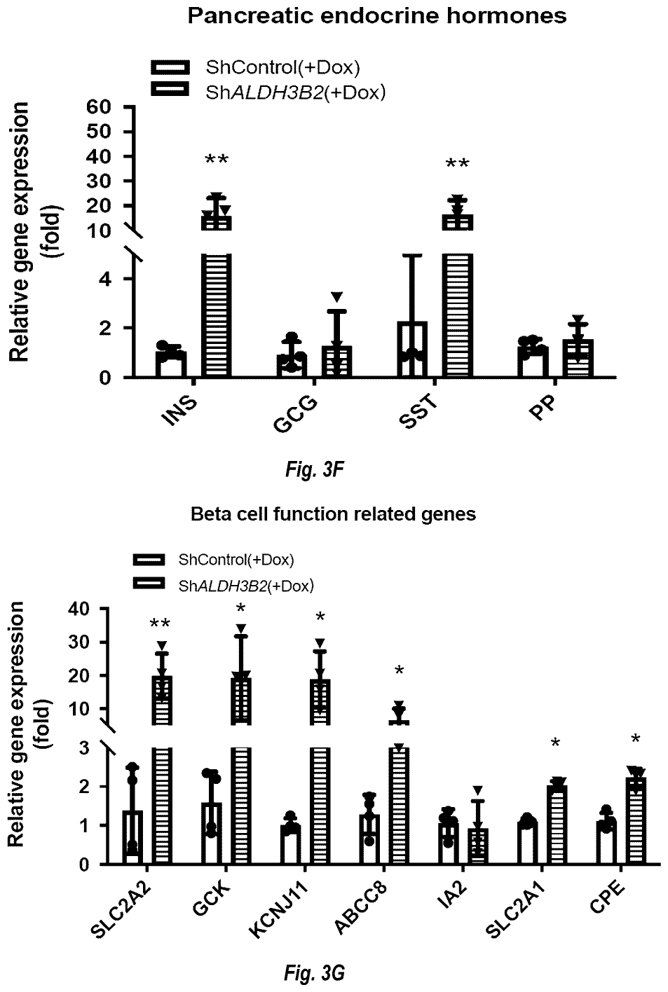

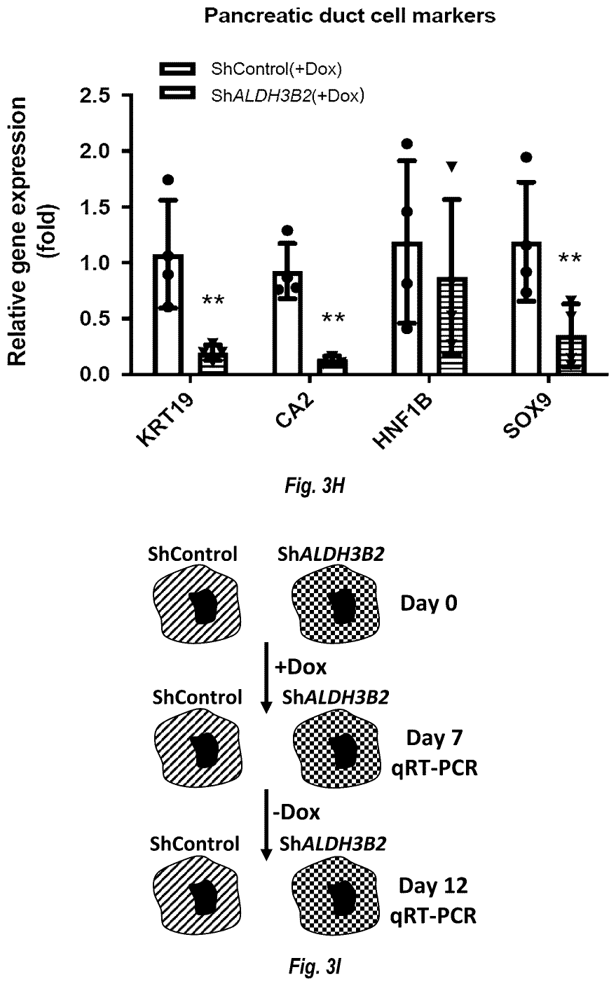

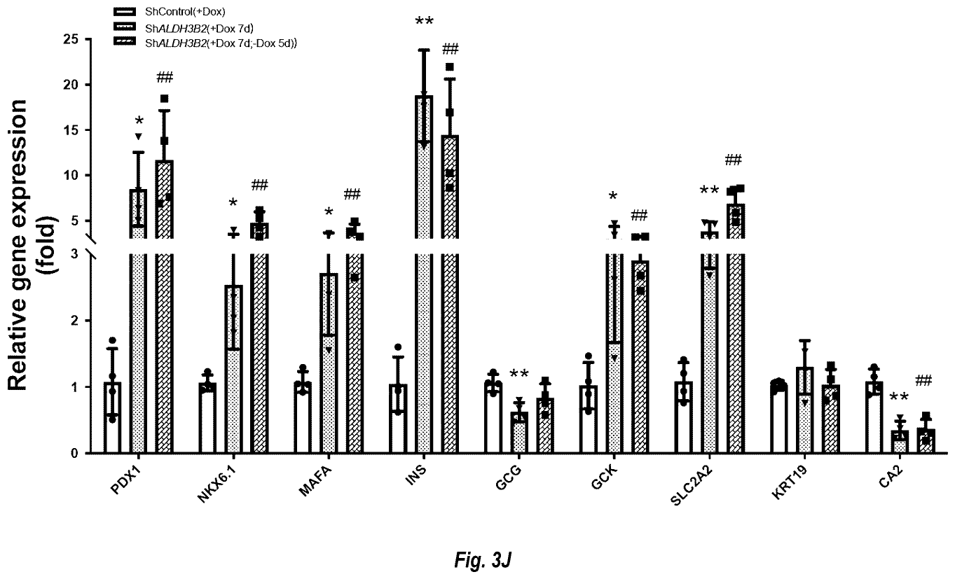

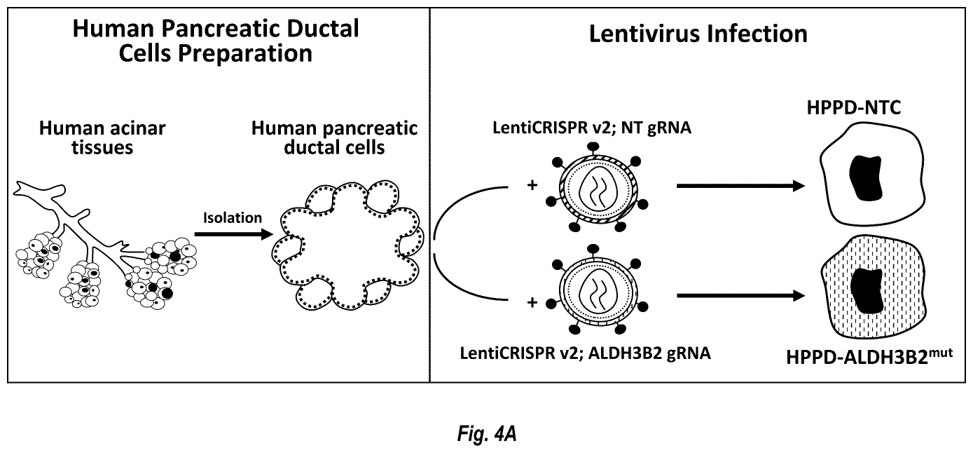

To ensure the mutation of ALDH3B2 induces bona fide cell transdifferentiation, rather than activating insulin expression only, a series of qPCR were performed to examine other pancreatic beta cell signature genes. The expression of several of key beta cell transcription factors, including Pdx1, MafA, Ngn3 and Pax6, were significantly upregulated in the ALDH3B2 mutant PANC-1 cells (FIG. 2F). The expression of pancreatic endocrine hormone, insulin (INS) and somatostatin (SST), but not glucagon (GCG) and pancreatic polypeptide (PP), were also increased in the ALDH3B2 mutant PANC-1 cells (FIG. 2G). The ALDH3B2 mutant PANC-1 cells have slightly decreased expression of pancreatic duct markers KRT19 and CA2, but not HNF1B or Sox9 (FIG. 3H). Genes that are critical for pancreatic beta cell function were also examined, and expression of Glut1 (SLC2A1), Glut2 (SLC2A2), Glucokinase (GCK), subunits of the ATP-sensitive potassium (K-ATP) (KCNJ11 and ABCC8), IA2, and CPE were all significantly upregulated in the ALDH3B2 mutant PANC-1 cells (FIG. 2I). In addition, using electron microscopy (EM), many of the ALDH3B2 mutant PANC-1 cells were found to have insulin granules, but no insulin granules were detected in the control NTC-PANC-1 cells (FIGS. 2J and 2K).

To evaluate if the ALDH3B2 mutation transdifferentiated beta-like cells are functional, the ALDH3B2 mutant or NTC PANC-1 cells were transplanted subcutaneously into streptozotocin (STZ) induced diabetic NSG mice (FIG. 2L). The mice transplanted with the ALDH3B2 mutant PANC-1 cells showed significantly improved glucose tolerance (FIG. 2M) and decreased daily random blood glucose (FIG. 2N) compared to the mice transplanted with NTC-PANC-1 cells. Notably, when the ALDH3B2 mutant PANC-1 graft was removed at the end of the study, the blood glucose of the mice elevated to the same level as in the control mice, confirming that the blood-glucose-lowering effect were indeed contributed by the transplanted ALDH3B2 mutant PANC-1 cells (FIG. 2N). The serum human insulin level was also significantly higher in the ALDH3B2 mutant PANC-1 cell transplanted mice (FIG. 2O).

An inducible short hairpin RNA (shRNA) system was used to ensure that the transdifferentiation of PANC-1 cell into beta-like cells by ALDH3B2 CRISPR knockout is indeed due to the loss-of-function of ALDH3B2, and not due to any off-targeting by the ALDH3B2 gRNA. A PANC-1 cell lines carrying a Tet-on inducible ALDH3B2 shRNA or a scrambled control shRNA was generated (FIG. 3A). The ALDH3B2 shRNA PANC-1 cells showed a significantly reduced ALDH3B2 expression after doxycycline treatment (FIGS. 3B and 3C). Similar to the ALDH3B2 CRISPR knockout PANC-1 cells, knocking down of ALDH3B2 by shRNA also transdifferentiated PANC-1 cells into beta-like cells. Human C-peptide expression was observed in shALDH3B2 PANC-1 cells by immunofluorescence (FIG. 3D). A series of qPCR experiments showed that the expression of key beta cell transcription factors (FIG. 3E), endocrine hormone insulin (INS) and somatostatin (SST) (FIG. 3F), and beta cell function related genes were significantly increased (FIG. 3G), whereas the expression of several pancreatic duct cell marker genes were reduced (FIG. 3H). In a separate experiment, ALDH3B2 expression was knocked down by treating the shALDH3B2 PANC-1 cells with doxycycline for 7 days, and subsequently removed doxycycline for another 5 days (FIG. 3I) to let the expression of ALDH3B2 to recover. The expression of beta cell signature genes was retained even after doxycycline withdrawal compared to right after doxycycline treatment, suggesting the transdifferentiation of PANC-1 cells by loss-of-function of ALDH3B2 is a stable cell fate change, and not only transient genes activation (FIG. 3J).

### Example 3. Transdifferentiation of Human Primary Pancreatic Duct Cells into Beta-Like Cells

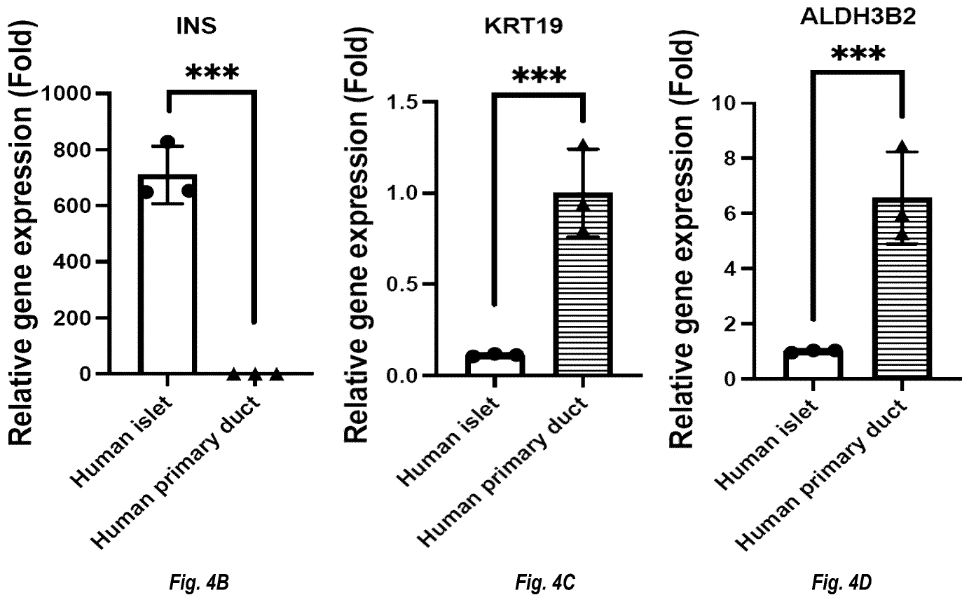

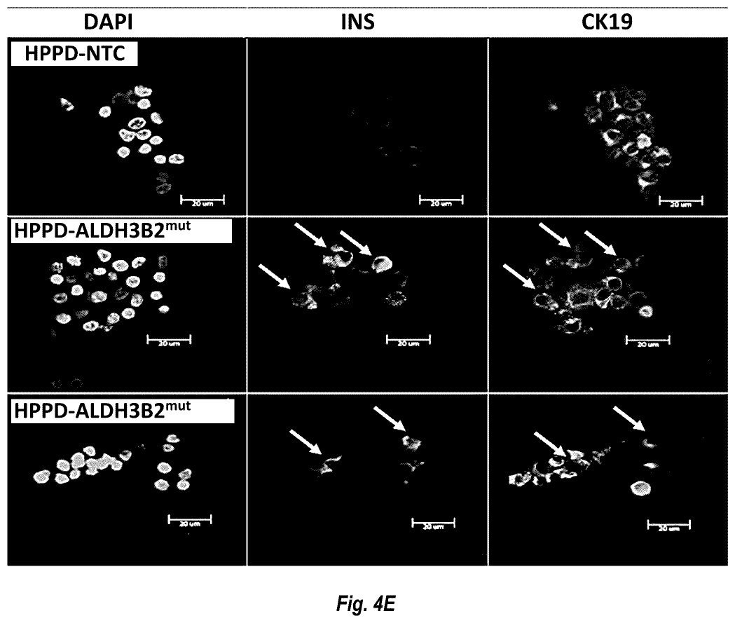

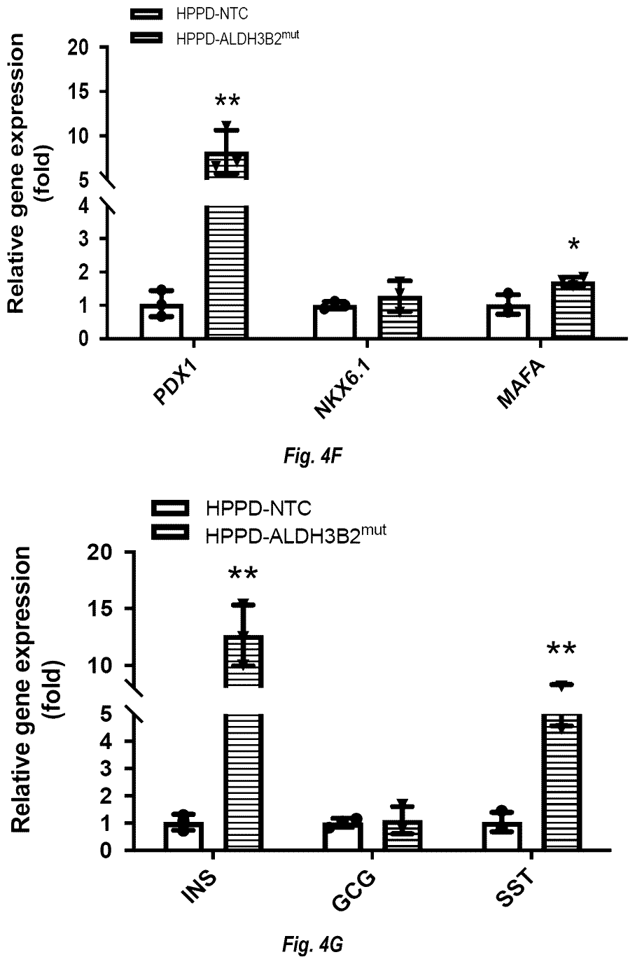

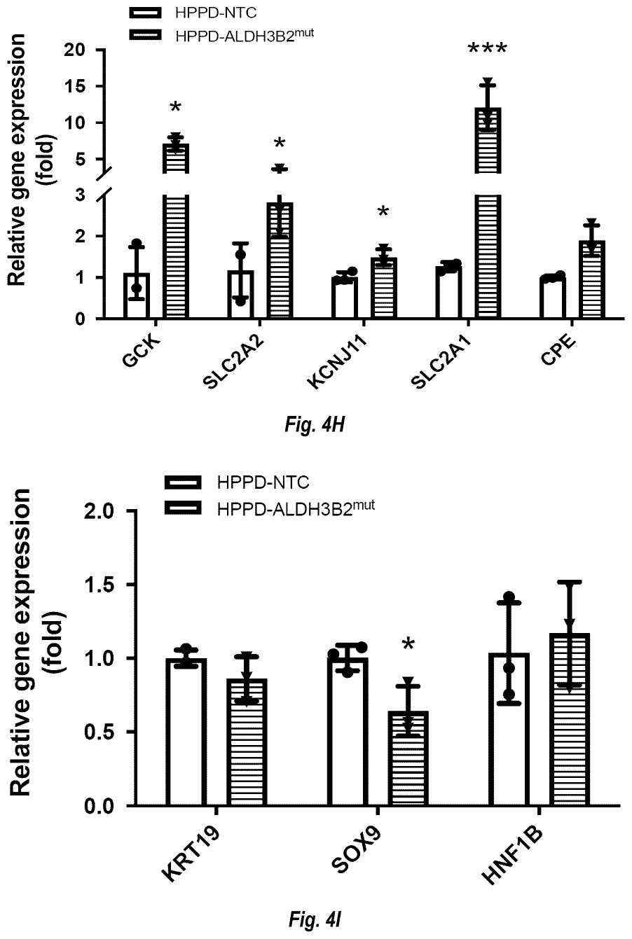

Next, disruption of ALDH3B2 was tested for the ability to transdifferentiate human primary pancreatic duct (HPPD) cells into beta-like cells similar to PANC-1 cells (FIG. 4A). Human primary duct cells were isolated and affinity-purified from human donor acinar tissues, and qPCR analysis revealed that there is no insulin expression (FIG. 4B), with significantly higher expression of pancreatic duct marker gene KRT19 (FIG. 4C) and ALDH3B2 (FIG. 4D) in the purified HPPD cells compared to primary human islets. The fact that human islets have a much lower ALDH3B2 expression level than HPPD cells (FIG. 4D) also supports the observation that deletion of ALDH3B2 in pancreatic duct cells leads to transdifferentiation into beta-like cells.

The purified HPPD cells were transduced by lentiviruses carrying SpCas9 and either ALDH3B2 gRNA (HPPD-ALDH3B2mut) or a non-targeting control gRNA (HPPD-NTC) (FIG. 4A). A portion of HPPD-ALDH3B2mut cells express human insulin while still retaining the CK19 expression, a signature of newly transdifferentiated beta cells during beta cell neogenesis, whereas in HPPD-NTC cells no insulin expression can be found in any cells (FIG. 4E). qPCR analysis showed that HPPD-ALDH3B2mut cells have significantly higher expression of key beta cell transcription factors (FIG. 4F), endocrine hormone insulin (INS) and Somatostatin (SST) (FIG. 4G) and beta cell function related genes (FIG. 4H), whereas the expression of several pancreatic duct cell marker genes were either unchanged or reduced (FIG. 4I).

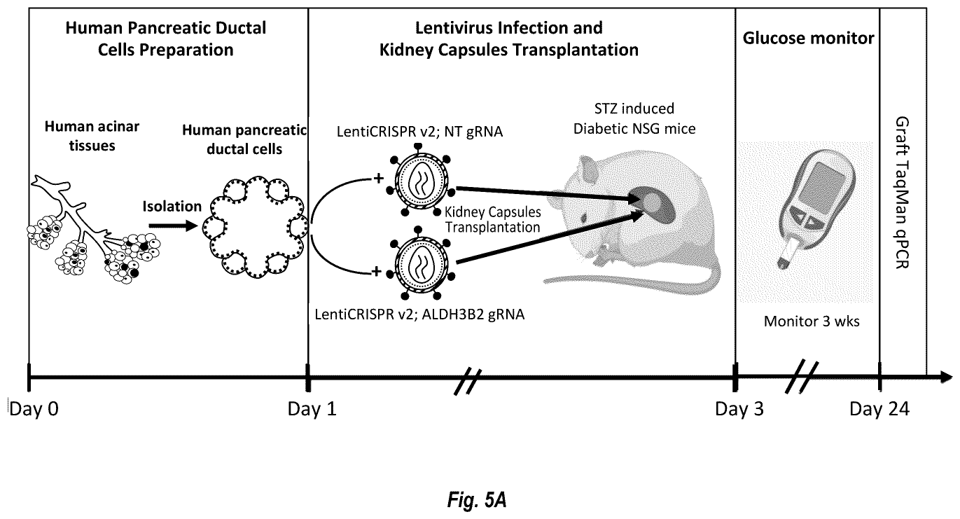

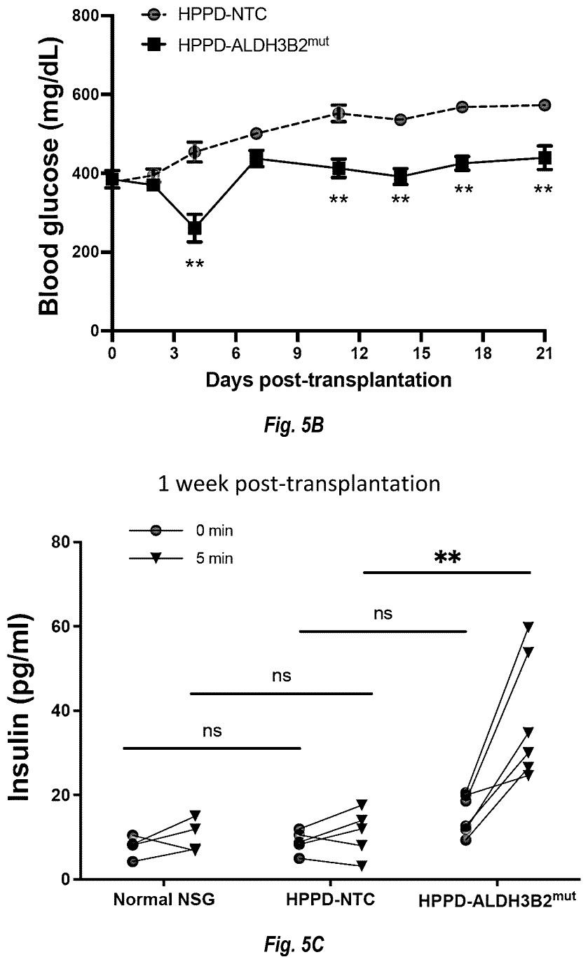

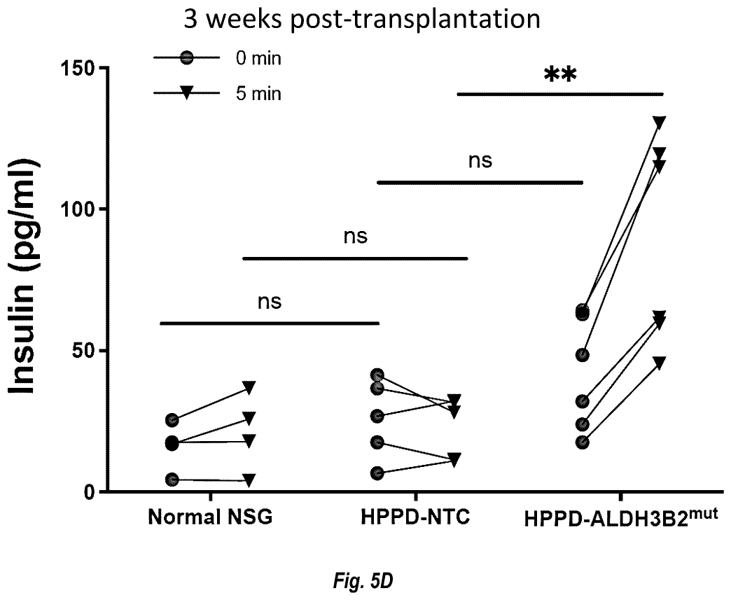

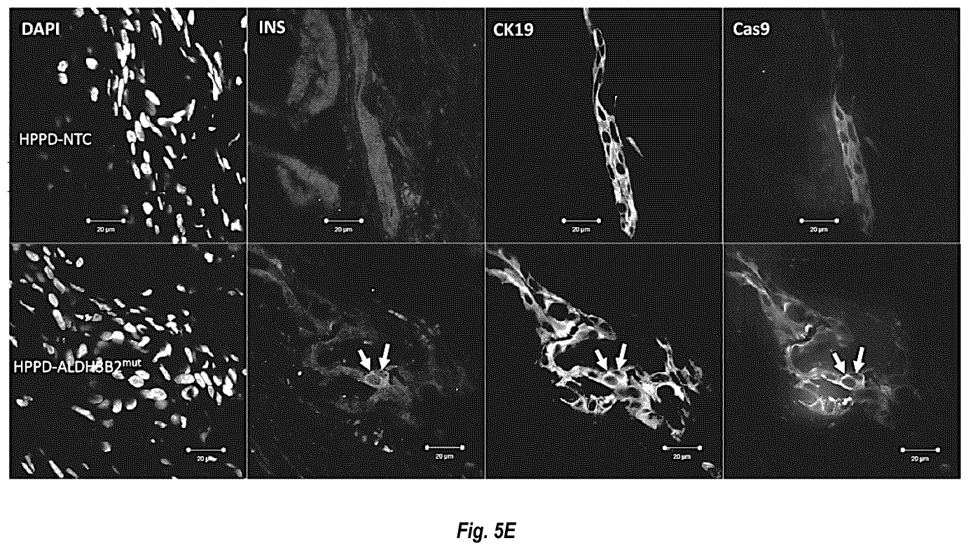

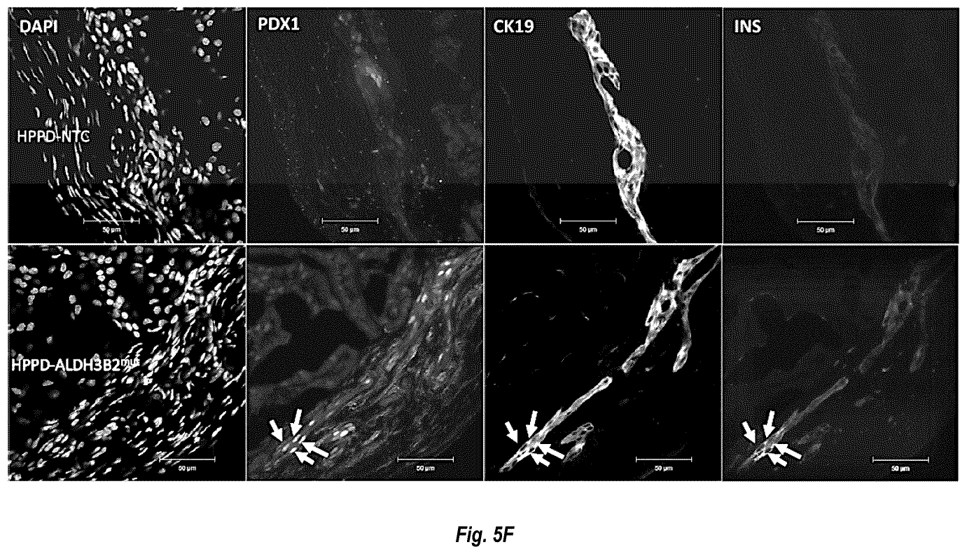

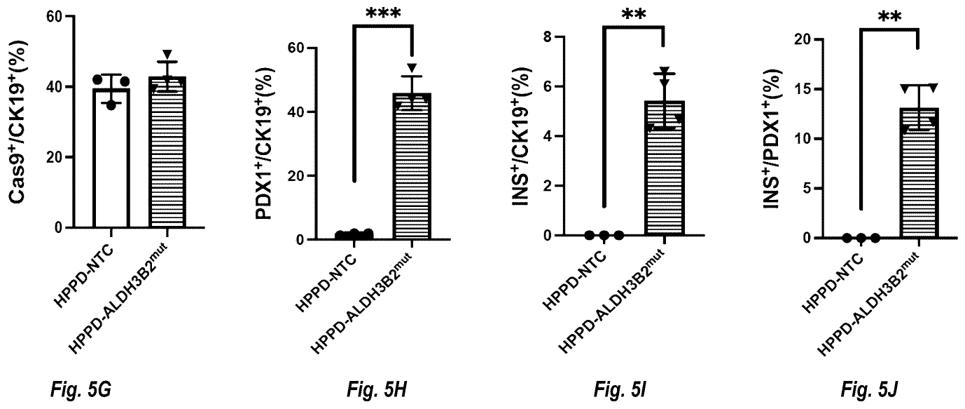

HPPD-ALDH3B2mut cells or HPPD-NTC cells were transplanted into the kidney capsule of streptozotocin (STZ) induced diabetic NSG mice, and then the function of the transplanted cells was evaluated by monitoring the blood glucose of the recipient mice (FIG. 5A). Mice transplanted with HPPD-ALDH3B2mut cells have significantly lower blood glucose compared to HPPD-NTC cells transplanted mice (FIG. 5B). More importantly, the transplanted HPPD-ALDH3B2mut cells can secrete human insulin in response to glucose, as significantly higher serum human insulin level was detected after 5 minutes of intraperitoneal injection of glucose (1 weeks or 3 weeks post-transplantation), whereas no such response was observed in HPPD-NTC cells transplanted mice (FIGS. 5C and 5D).

Histological analysis revealed that insulin+/CK19+ cells only exist in the transplanted HPPD-ALDH3B2mut cells but not the HPPD-NTC cells (FIG. 5E). About 40% of the transplanted HPPD-NTC cells or HPPD-ALDH3B2mut cells were successfully infected with the NT or ALDH3B2 gRNA lentivirus (shown by quantification of the percentage of Cas9+/CK19+ cells, FIG. 5F), and among all the gRNA lentivirus infected pancreatic duct cells, ˜15% of HPPD-ALDH3B2mut cells express human insulin (FIG. 5G). Interestingly, the majority of the pancreatic duct cells were infected with ALDH3B2 gRNA lentivirus co-express Pdx1 and CK19 (FIGS. 5H and 5I), and around 12% of the Pdx1+ cells express human insulin (FIG. 5J). Co-expression of Pdx1 and CK19 is a signature of pancreatic progenitor cells²¹, so it is likely that ALDH3B2 mutation de-differentiate the mature pancreatic duct cells into pancreatic progenitor cells, and then portion of the progenitor cells spontaneously differentiate into beta-like cells.

### Example 4. Example 4. Epigenetic Changes Due to ALDH3B2 Inhibition

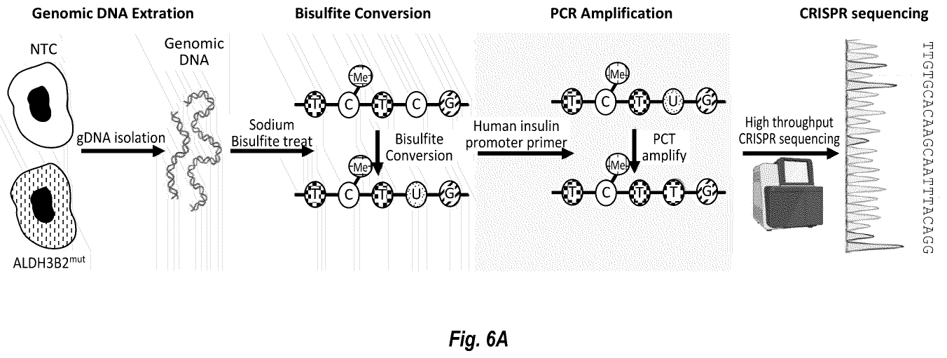

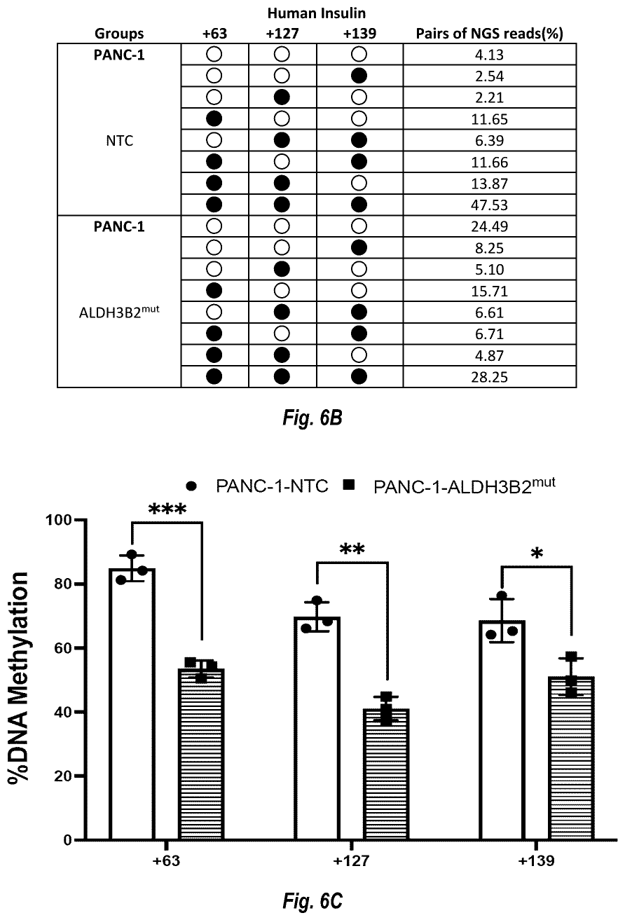

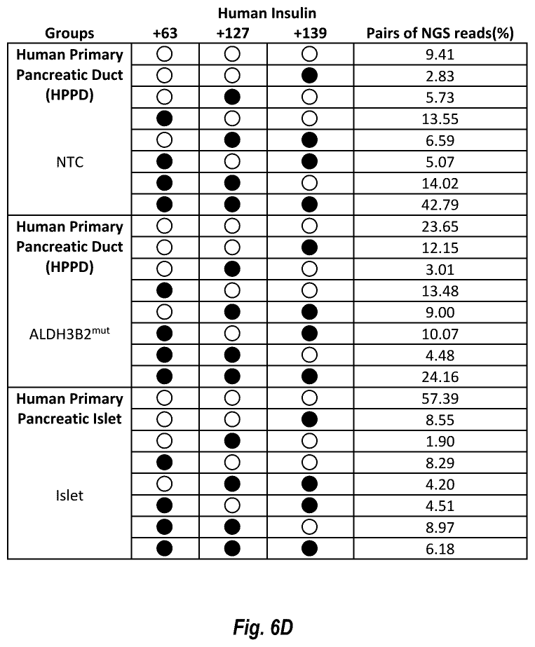

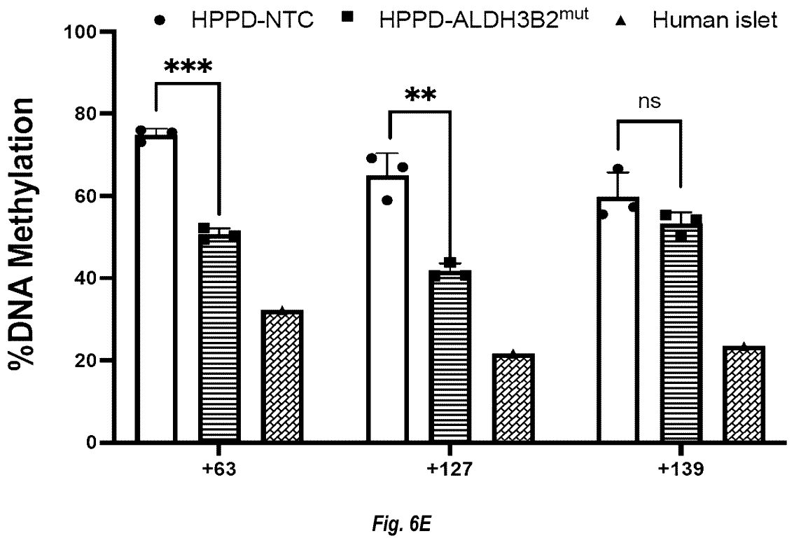

Since ALDH3B2 loss-of-function led to stable cell fate change from pancreatic duct cells to beta-like cell (FIGS. 3I and 3J), epigenetic changes in ALDH3B2 mutant PANC-1 cells and primary pancreatic duct cells were evaluated. Specifically, the DNA methylation in the human insulin gene region was evaluated by bisulfite conversion assay (FIG. 6A). Indeed, DNA methylation was greatly reduced in the ALDH3B2 mutant PANC-1 cells compared to control NTC PANC-1 cells at +63, +127 and +139 position of the human insulin locus, which are three common DNA methylation sites for human insulin locus shown by previous reports²² (FIGS. 6B and 6C). In the DNA methylation analysis of the primary human pancreatic duct cells, human primary pancreatic islets were included for comparison, and ALDH3B2 mutation also significantly reduced the DNA methylation at the three sites in the human insulin gene locus (FIGS. 6D and 6E). Although the ALDH3B2 mutation did not reduce the DNA methylation to the level of human primary pancreatic islets, only a portion of pancreatic duct cells may have transdifferentiated into beta-like cells with ALDH3B2 mutation. Overall, the DNA methylation analysis suggests that loss-of-function of ALDH3B2 was able to induce epigenetic changes in the pancreatic duct cells and hence induce bonafide cell fate change into pancreatic beta-like cells.

In summary, the present unbiased and genome-wide search looked for genes whose loss-of-function would transdifferentiate pancreatic duct cells into insulin-producing beta-like cells. Mutation or suppression of a single gene, ALDH3B2, in pancreatic duct cells is sufficient to induce bonafide cell fate change from pancreatic duct cells to beta-like cells. Although the pancreatic beta cell neogenesis in human is frequently observed evidenced by existence of INS*/CK19+ cells in pancreatic ductal epithelium, it is still a relatively rare event, as the percentage of INS' pancreatic duct cells in human is only at the level of 1%²³. Here, disruption of ALDH3B2 was able to transdifferentiate human pancreatic duct cells into beta-like cells with a high efficiency of ˜15%, which provides strong hope to harness the potential of beta cell neogenesis for human beta cell mass replenishment.

It is not yet well understood what exactly the function of ALDH3B2 is in the cells. From limited mouse studies, it was shown that ALDH3B2 localizes on lipid droplet in cells and catalyze long-chain fatty aldehyde into long-chain fatty acid²⁴, but it is unknown whether long-chain fatty acids/aldehydes may play a role in the pancreatic duct cell transdifferentiation into beta-like cells, which warrant further studies. Nevertheless, the discovery of ALDH3B2 as a regulator of pancreatic beta cells neogenesis provides a promising therapeutic target for pancreatic beta cell mass restoration. The endogenous pancreatic duct cells could be potentially targeted by gene-editing to edit ALDH3B2 and induce transdifferentiation. Alternatively, it is plausible to target ALDH3B2 enzymatic activity by small molecules in order to achieve the same effect as genetic disruption of ALDH3B2 gene. It should be noted that the whole study was conducted using human pancreatic duct cell line or primary human pancreatic duct cells, so the findings here may have a higher possibility of translating into human diabetes therapeutics.

### Example 5. Materials and Methods

The following methods were used in the experiments.

### Mice

NSG (NOD.Cg-Prkd^{scid} Il2rg^{tm1Wj1}/SzJ) mice were purchased from the Jackson Laboratory (Bar Harbor, ME). Animals were housed in pathogen-free facilities at the Joslin Diabetes Center, and all experimental procedures were approved and performed in accordance with institutional guidelines and regulations.

### REPB Reporter Construction and REPB PANC-1 Cell Generation

The REPB reporter lentivirus vector was constructed by assembling the rat insulin promoter, RIP3.1 promoter²⁵, EGFP, and Blasticidin-S deaminase (BSD). The EGFP and BSD genes are fused together with P2A peptide. The REPB reporter lentivirus was used to infect PANC-1 cells (ATCC #CRL-1469), and the infected PANC-1 cells were then single-cell sorted by FACS. PANC-1 clones with confirmed REPB reporter genome incorporation were used in the genome-wide CRISPR screen.

### CRISPR GeCKO Library Screen

The human GeCKO-v2 (Genome-Scale CRISPR Knock-Out) lentiviral pooled library was obtained from Addgene (Addgene, #1000000048) and was prepared as previously described²⁶. 100 million REPB PANC-1 cells, which carry on REPB promoter were infected with human GeCKO CRISPR lentiviral library at MOI=0.3, and then selected by puromycin (2.5 μg/ml) at day 3 post-lentivirus infection. After cells recover from puromycin selection, 20 million cells were collected as baseline control (CON-1 and CON-2), the rest of the cells were selected by blasticidin (10 μg/ml) for 7 days. The blasticidin-resistant cells were allowed to grow back to full confluence and subjected to FACS sorting based on their EGFP intensity. Cell population with the highest EGFP intensity were collected as experiment group (EXP-1 and EXP-2). Genomic DNA was extracted from the cells (Quick-gDNA midiprep kit, Zymo Research), the NGS (Next Generation Sequencing) libraries were prepared as previously described²⁷, and subjected to NGS sequencing analysis (Novogene, CA). The gRNA sequences from the NGS sequencing data were extracted using standard bioinformatics methods, and the distribution of gRNAs were calculated as Count Per Million (CPM).

### Cell Lines

PANC-11 (#CRL-1469) and 293FT (#R7007) cell lines were obtained from ATCC and Thermo Fisher Scientific, respectively. Cells were maintained in DMEM (Gibco, 10313039), supplemented with 10% fetal bovine serum (FBS, Gibco), L-alanyl-L-glutamine (Gibco) and penicillin/streptomycin (Corning), in a 37° C. incubator with 5% CO₂. To generate non-targeting control (NTC) and ALDH3B2^{mut} PANC-1 cells, non-targeting (NT) gRNA (5′-GCTTTCACGGAGGTTCGACG-3′, SEQ ID NO: 1) or ALDH3B2 gRNA (5′-GCCCTCCTCACCTGCGGCGA-3′, HGLibA_01571, SEQ ID NO: 2) oligonucleotides were cloned into LentiCRISPR-v2 vector. Wild type PANC-1 cells were then transduced by NTC or ALDH3B2 gRNA-containing lentivirus, and subsequently selected by puromycin treatment. Indel mutation in ALDH3B2^{mut} cells was confirmed by deep sequencing analysis (MGH DNA Core Facility, Cambridge, MA). All plasmid sequences were verified by Sanger sequencing before transduction and transfection. To generate shControl and shALDH3B2 PANC-1 cells, a scrambled or ALDH3B2-targeting shRNA was cloned into FH1t(INSR)UTG-GFP vector (a gift from Dr. Stephan Kissler). shControl and shALDH3B2 lentiviruses were then used to infect PANC-1 cells and the cells were selected by puromycin treatment.

### Human Pancreatic Ductal Cell Isolation and Purification

Human primary pancreatic ductal cells isolation was performed as previously described^{28, 29}. In brief, human acinar tissue from Integrated Islet Distribution Program (IIDP) was washed 2 times with PBS, and then incubated with trypsin solution (1.5 ml 0.25% Trypsin in 20 ml PBS) shaking at 37° C. for 15 minutes. Dispersed cells were centrifuged at 1,000 rpm for 5 minutes, the supernatant was aspirated, and then the pellets were resuspended with mouse antihuman CA19-9 antibody (Invitrogen; clone 116-NS-19-9) in 2 ml PBS solution. After 15-minute incubation at 4° C., the cell suspension was mixed with 10 ml PBS solution (375 mg EDTA and 2.5 g BSA in 500 ml PBS) gently. Tubes were centrifuged at 1,000 rpm for 5 minutes, supernatant was aspirated, 250 μl/tube goat anti-mouse IgG microbeads (Miltenyi Biotec) in PBS solution were added, and pellets were mixed. After 20 minutes incubation at 4° C., pellets were wash with PBS solution 2 times. Tubes were centrifuged at 1,000 rpm for 5 minutes, and pellets were resuspended in 20 ml cold PBS solution and passed through 40 m cell strainers to remove newly formed clumps of cells. MACS magnetic LS separation columns (Miltenyi Biotec) were prepared according to the manufacturer's instructions.

### Preparation and Transplantation of Primary Human Pancreatic Duct Cells

Human primary pancreatic ductal cells isolation was performed as described above. Purified human primary pancreatic ductal cells (HPPD) were immediately cultured in a low-attachment plate in RMPI DMEM/F12 medium (Gibco), supplemented with 10% FBS and penicillin/streptomycin. Lentivirus encoding a NTC or ALDH3B2 gRNA together with Cas9 endonuclease was added to the culture media for overnight infection. The next day, HPPD cells were washed with culture media twice and ˜10⁷ cells were transplanted under the left kidney capsule of 8-week-old of STZ induced diabetic male NSG mice. Graft recipients were left to recover from surgery for three weeks. At day 21 post-cell-transplantation, grafts were retrieved for gene expression analysis by quantitative real-time PCR (qPCR).

Quantitative Real-Time PCR (qPCR)

Cells or grafts were treated with TRIzol (Thermo Fisher Scientific) for RNA extraction following the manufacturer's protocol. Purified RNA was reverse-transcribed into cDNA using the SuperScript IV first-strand synthesis kit (Invitrogen). INS (Hs00355773_m1), GCG (Hs01031536_m1), SST(Hs00356144_m1) PDXI(Hs00236830_m1), NKX6-1 (Hs00232355_m1), GCK (Hs01564555_m1), SLC2A2 (Hs00165775_m1), SLC2A1 (Hs00892681_m), CPE (Hs00960598_m1), CA2 (Hs01070108_m1), KRT19 (Hs00761767_s1), SOX9 (Hs00165814_m1), ALDH3B2 (Hs02511514_s1) and ACTB (Hs01060665_g1) probes for TaqMan assays were purchased from Thermo Fisher Scientific. All Gene expression levels were analyzed by SYBR green PowerUp qPCR assays (Applied Biosystems).

Primer sequences used for ACTB: forward—5′ CACCATTGGCAATGAGCGGTTC 3′ (SEQ ID NO: 3); reverse—5′AGGTCTTTGCGGATGTCCACGT 3′ (SEQ ID NO: 4); INS: forward—5′ ACGAGGCTTCTTCTACACACCC 3′ (SEQ ID NO: 5); reverse—5′ TCCACAATGCCACGCTTCTGCA 3′ (SEQ ID NO: 6); PDXJ: forward—5′GAAGTCTACCAAAGCTCACGCG 3′ (SEQ ID NO: 7); reverse—5′ GGAACTCCTTCTCCAGCTCTAG 3′ (SEQ ID NO: 8); NKX6.1: forward—5′ CCTATTCGTTGGGGATGACAGAG 3′ (SEQ ID NO: 9); reverse—5′ TCTGTCTCCGAGTCCTGCTTCT 3′ (SEQ ID NO: 10); PAX6: forward—5′ CTGAGGAATCAGAGAAGACAGGC 3′ (SEQ ID NO: 11); reverse—5′ ATGGAGCCAGATGTGAAGGAGG 3′ (SEQ ID NO: 12); MAFA: forward—5′ GCTTCAGCAAGGAGGAGGTCAT 3′ (SEQ ID NO: 13); reverse—5′ TCTGGAGTTGGCACTTCTCGCT 3′ (SEQ ID NO: 14); NEUROD: forward—5′ GGTGCCTTGCTATTCTAAGACGC 3′ (SEQ ID NO: 15); reverse—5′ GCAAAGCGTCTGAACGAAGGAG 3′ (SEQ ID NO: 16); NGN3: forward—5′ CCTAAGAGCGAGTTGGCACTGA 3′ (SEQ ID NO: 17); reverse—5′ AGTGCCGAGTTGAGGTTGTGCA 3′ (SEQ ID NO: 18); GCG: forward—5′ CGTTCCCTTCAAGACACAGAGG 3′ (SEQ ID NO: 19); reverse—5′ ACGCCTGGAGTCCAGATACTTG 3′ (SEQ ID NO: 20); SST: forward—5′ CCAGACTCCGTCAGTTTCTGCA 3′ (SEQ ID NO: 21); reverse—5′ TTCCAGGGCATCATTCTCCGTC 3′ (SEQ ID NO: 22); PP: forward—5′ AGACACAAAGAGGACACGCTGG 3′ (SEQ ID NO: 23); reverse—5′ GAGTCGTAGGAGACAGAAGGTG 3′ (SEQ ID NO: 24); SLC2A2: forward—5′ ATGTCAGTGGGACTTGTGCTGC 3′ (SEQ ID NO: 25); reverse—5′ AACTCAGCCACCATGAACCAGG 3′ (SEQ ID NO: 26); GCK: forward—5′ CATCTCCGACTTCCTGGACAAG 3′ (SEQ ID NO: 27); reverse—5′ TGGTCCAGTTGAGAAGGATGCC 3′ (SEQ ID NO: 28); ABCC8: forward—5′ GACGACAAGAGGACAGTGGTCT 3′ (SEQ ID NO: 29); reverse—5′ GCATTCAGACCTCTGGAAGTCC 3′ (SEQ ID NO: 30); KCNJ11: forward—5′ TGTGTCACCAGCATCCACTCCT 3′ (SEQ ID NO: 31); reverse—5′ GTTCTGCACGATGAGGATCAGG 3′ (SEQ ID NO: 32); IA2: forward—5′ TGGAGATCCTGGCTGAGCATGT 3′ (SEQ ID NO: 33); reverse—5′ GGTCACATCAGCCAAAGACAGG 3′ (SEQ ID NO: 34); KRT19: forward—5′ AGCTAGAGGTGAAGATCCGCGA 3′ (SEQ ID NO: 35); reverse—5′ GCAGGACAATCCTGGAGTTCTC 3′ (SEQ ID NO: 36); CA2: forward—5′ GTGACCTGGATTGTGCTCAAGG 3′ (SEQ ID NO: 37); reverse—5′ GTTGTCCACCATCAGTTCTTCGG 3′ (SEQ ID NO: 38); HNF1B: forward—5′ CCCAGCAAATCTTGTACCAGGC 3′ (SEQ ID NO: 39); reverse—5′ ACCTCAGTGACCAAGTTGGAGC 3′ (SEQ ID NO: 40); and SOX9: forward—5′ AGGAAGCTCGCGGACCAGTAC 3′ (SEQ ID NO: 41); reverse—5′ GGTGGTCCTTCTTGTGCTGCAC 3′ (SEQ ID NO: 42). All qPCR assays were performed using a QuantStudio 6 Flex Real-Time PCR system (Applied Biosystems).

### Western Blotting

Cell lysates were collected on ice in RIPA lysis buffer containing proteinase and phosphatase inhibitors (complete proteinase inhibitor cocktail, Sigma-Aldrich; Pierce phosphatase inhibitor, Thermo Scientific Fisher). Protein concentrations were measured by Pierce BCA protein assay (Thermo Scientific Fisher). 40 μg denatured cell lysate protein was used for SDS-PAGE electrophoresis (4-20% TGX gel, Bio-Rad). The following primary antibodies were used: ALDH3B2 (Proteintech, #15746-1-AP) and actin (ABclonal, #AC004). All images were obtained and quantified using a C-DiGit blot scanner and the Image Studio software (LI-COR Biosciences).

### Transplantation Studies

Experimental diabetes was induced in 8-week-old NSG male mice by intraperitoneal injection of streptozotocin (STZ) (40 mg STZ/kg body weight for five consecutive days). Animals were considered diabetic only if morning-fed blood glucose exceeded 350 mg/dl. Three days after STZ injection, ˜10⁷ PANC-1 cells (carry NT and ALDH3B2^{mut}) were transplanted subcutaneously into each diabetic NSG mouse. Blood glucose was monitored every 3-4 days. Six weeks post cells transplantation, Intraperitoneal glucose tolerance test (IPGTT) was performed. Mice were fasted for 16 hours. The plasma glucose levels of the mice before (baseline) or 15, 30, 60, 90, and 120 minutes after intraperitoneal injection of 2 mg/g body weight glucose were recorded by a glucose meter. At day 52 post cells transplantation, surgically remove the grafts from NSG mice, measured the blood glucose 4 days later.

### Immunofluorescence Staining and Confocal Microscopy

The grafts with kidney were dissected from the mice, tissue fixed for 1 hour in 4% paraformaldehyde at 4° C. and dehydrated using 30% sucrose solution overnight. The tissues were embedded in disposable base molds (Thermo Fisher Scientific), and 10 mm sections were cut. For staining, slides were blocked with PBS+0.1% Triton X-100 (Thermo Fisher Scientific)+5% donkey serum (Sigma-Aldrich) for 1 hour at room temperature (RT), incubated with primary antibodies overnight at 4° C., washed, incubated with secondary antibody incubation for 1 hour at RT, incubated with Hoechst 33342 (Invitrogen) for 10 minutes at RT, and washed. For imaging, samples were mounted in fluorescence mounting medium (Dako), covered with coverslips, and sealed with nail polish. Representative images were taken using a Zeiss LSM 710 confocal microscope. Primary antibody was Insulin (A0564, Dako), Pdx1 (5679S, Cell Signaling Technology), Dykddddk Tag (14793S, Cell Signaling Technology), C-peptide (GN-ID4, Developmental Studies Hybridoma Bank), or Cytokeratin 19 (Abcam, ab7754).

Cells were seeded into 4-well culture slide (Falcon) at density of 10⁵ cells/well. After another 24 hours, the cells were fixed, stained, and subjected to fluorescence microscopic analysis as above described.

### Proinsulin and Insulin Content Measurement

Acid-ethanol extraction was used to extract protein, and proinsulin and insulin contact measured using total human proinsulin (Alpco) and STELLUX® Chemi Human Insulin ELISA kits (Alpco).

### Electron Microscopy

To analyze granular ultrastructure, PANC-1 cells carrying NT or ALDH3B2^{mut} were fixed at RT for 2 hours with a mixture containing 1.25% PFA, 2.5% glutaraldehyde, and 0.03% picric acid in 0.1M sodium cocodylate buffer (pH 7.4). Samples were then sent to Advanced Microscopy Core of Joslin for further operating and transmission electron microscope imaging.

### DNA Methylation Analyses

Bisulfite conversion (Zymo Research, EZ DNA Methylation-Direct Kits) of DNA from PANC-1, human primary pancreatic ductal cells carry NT or ALDH3B2^{mut} and human islets were performed as described previously³⁰. Bisulfite-treated DNA was PCR amplified, using primers (human insulin promoter forward primer—5′ AGGATAGGTTGTATTAGAAGAGGTTATTAAG 3′ (SEQ ID NO: 43); human INS promoter reverse primer-5′ CCCCTAAACTCACCCCCACATACTTC 3′ (SEQ ID NO: 44)) specific for bisulfite treated DNA but independent of methylation status at monitored CpG sites. Reaction conditions for the first round of PCR were 5 cycles of 95° C. 1 minute, 52° C. 3 minutes, 72° C. 3 minutes followed by 40 cycles of 95° C. 30 seconds, 55° C. 45 seconds, 72° C. 45 seconds, and followed by 7 minutes at 72° C. PCR products were gel purified and used for deep sequencing analysis (MGH DNA Core Facility, Cambridge, MA).

### Statistical Analyses

Statistical analyses were performed by unpaired or paired tests as indicated using the Prism 8 software. All data are presented as mean±SEM. Significance was defined as *p<0.05, **p<0.01 and ***p<0.001. No samples were excluded from the analysis. Data analysis was not blinded. All data are representative of two or more similar experiments.

### REFERENCES

1 Weir, G. C., Bonner-Weir, S. & Leahy, J. L. Islet mass and function in diabetes and transplantation. Diabetes 39, 401-405, doi:10.2337/diab.39.4.401 (1990).
2 Nair, G. & Hebrok, M. Islet formation in mice and men: lessons for the generation of functional insulin-producing beta-cells from human pluripotent stem cells. Curr Opin Genet Dev 32, 171-180, doi:10.1016/j.gde.2015.03.004 (2015).
3 Nostro, M. C. et al. Efficient generation of NKX6-1+ pancreatic progenitors from multiple human pluripotent stem cell lines. Stem Cell Reports 4, 591-604, doi:10.1016/j.stemcr.2015.02.017 (2015).
4 Pagliuca, F. W. et al. Generation of functional human pancreatic beta cells in vitro. Cell 159, 428-439, doi:10.1016/j.cell.2014.09.040 (2014).
5 Shapiro, A. M. et al. Islet transplantation in seven patients with type 1 diabetes mellitus using a glucocorticoid-free immunosuppressive regimen. N Engl J Med 343, 230-238, doi:10.1056/NEJM200007273430401 (2000).
6 Zhou, Q. & Melton, D. A. Pancreas regeneration. Nature 557, 351-358, doi:10.1038/s41586-018-0088-0 (2018).
7 Ben-Othman, N. et al. Long-Term GABA Administration Induces Alpha Cell-Mediated Beta-like Cell Neogenesis. Cell 168, 73-85 e11, doi:10.1016/j.cell.2016.11.002 (2017).
8 Collombat, P. et al. The ectopic expression of Pax4 in the mouse pancreas converts progenitor cells into alpha and subsequently beta cells. Cell 138, 449-462, doi:10.1016/j.cell.2009.05.035 (2009).
9 Zhou, Q., Brown, J., Kanarek, A., Rajagopal, J. & Melton, D. A. In vivo reprogramming of adult pancreatic exocrine cells to beta-cells. Nature 455, 627-632, doi:10.1038/nature07314 (2008).
10 Aguayo-Mazzucato, C. & Bonner-Weir, S. Pancreatic beta Cell Regeneration as a Possible Therapy for Diabetes. Cell Metab 27, 57-67, doi:10.1016/j.cmet.2017.08.007 (2018).
11 Bonner-Weir, S., Aguayo-Mazzucato, C. & Weir, G. C. Dynamic development of the pancreas from birth to adulthood. Ups J Med Sci 121, 155-158, doi:10.3109/03009734.2016.1154906 (2016).
12 Bonner-Weir, S. et al. The pancreatic ductal epithelium serves as a potential pool of progenitor cells. Pediatr Diabetes 5 Suppl 2, 16-22, doi:10.1111/j.1399-543X.2004.00075.x (2004).
13 Inada, A. et al. Carbonic anhydrase II-positive pancreatic cells are progenitors for both endocrine and exocrine pancreas after birth. Proc Natl Acad Sci USA 105, 19915-19919, doi:10.1073/pnas.0805803105 (2008).
14 Butler, A. E. et al. Adaptive changes in pancreatic beta cell fractional area and beta cell turnover in human pregnancy. Diabetologia 53, 2167-2176, doi:10.1007/s00125-010-1809-6 (2010).
15 Lieber, M., Mazzetta, J., Nelson-Rees, W., Kaplan, M. & Todaro, G. Establishment of a continuous tumor-cell line (panc-1) from a human carcinoma of the exocrine pancreas. Int J Cancer 15, 741-747, doi:10.1002/ijc.2910150505 (1975).
16 Madden, M. E. & Sarras, M. P., Jr. Morphological and biochemical characterization of a human pancreatic ductal cell line (PANC-1). Pancreas 3, 512-528, doi:10.1097/00006676-198810000-00003 (1988).
17 Sanjana, N. E., Shalem, O. & Zhang, F. Improved vectors and genome-wide libraries for CRISPR screening. Nat Methods 11, 783-784, doi:10.1038/nmeth.3047 (2014).
18 Marchitti, S. A., Brocker, C., Stagos, D. & Vasiliou, V. Non-P450 aldehyde oxidizing enzymes: the aldehyde dehydrogenase superfamily. Expert Opin Drug Metab Toxicol 4, 697-720, doi:10.1517/17425255.4.6.697 (2008).
19 Vassalli, G. Aldehyde Dehydrogenases: Not Just Markers, but Functional Regulators of Stem Cells. Stem Cells Int 2019, 3904645, doi:10.1155/2019/3904645 (2019).
20 Kim-Muller, J. Y. et al. Aldehyde dehydrogenase 1a3 defines a subset of failing pancreatic beta cells in diabetic mice. Nat Commun 7, 12631, doi:10.1038/ncomms12631 (2016).
21 Wescott, M. P. et al. Pancreatic ductal morphogenesis and the Pdx1 homeodomain transcription factor. Mol Biol Cell 20, 4838-4844, doi:10.1091/mbc.E09-03-0203 (2009).
22 Neiman, D. et al. Islet cells share promoter hypomethylation independently of expression, but exhibit cell-type-specific methylation in enhancers. Proc Natl Acad Sci USA 114, 13525-13530, doi:10.1073/pnas.1713736114 (2017).
23 Mezza, T. et al. Insulin resistance alters islet morphology in nondiabetic humans. Diabetes 63, 994-1007, doi:10.2337/db13-1013 (2014).
24 Kitamura, T., Takagi, S., Naganuma, T. & Kihara, A. Mouse aldehyde dehydrogenase ALDH3B2 is localized to lipid droplets via two C-terminal tryptophan residues and lipid modification. Biochem J 465, 79-87, doi:10.1042/BJ20140624 (2015).
25 Chai, R., Chen, S., Ding, J. & Grayburn, P. A. Efficient, glucose responsive and islet-specific transgene expression by a modified rat insulin promoter. Gene Ther 16, 1202-1209, doi:10.1038/gt.2009.114 (2009).
26 Joung, J. et al. Genome-scale CRISPR-Cas9 knockout and transcriptional activation screening. Nat Protoc 12, 828-863, doi:10.1038/nprot.2017.016 (2017).
27 Shalem, O. et al. Genome-scale CRISPR-Cas9 knockout screening in human cells. Science 343, 84-87, doi:10.1126/science.1247005 (2014).
28 Corritore, E. et al. beta-Cell differentiation of human pancreatic duct-derived cells after in vitro expansion. Cell Reprogram 16, 456-466, doi:10.1089/cell.2014.0025 (2014).
29 Yatoh, S. et al. Differentiation of affinity-purified human pancreatic duct cells to beta-cells. Diabetes 56, 1802-1809, doi:10.2337/db06-1670 (2007).
30 Millar, D. S., Warnecke, P. M., Melki, J. R. & Clark, S. J. Methylation sequencing from limiting DNA: embryonic, fixed, and microdissected cells. Methods 27, 108-113, doi:10.1016/s1046-2023(02)00061-0 (2002).

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

1. A method of inducing insulin production from pancreatic duct cells in response to glucose comprising administering an agent that inhibits ALDH3B2 protein or an agent that inhibits the expression of ALDH3B2 gene.

2. The method of claim 1, wherein the insulin production in response to glucose is maintained after withdrawing the agent.

3. A method of transdifferentiating pancreatic duct cells into insulin-producing beta-like cells comprising administering an agent that inhibits ALDH3B2 protein or an agent that inhibits the expression of ALDH3B2 gene.

4. The method of claim 3, wherein:
a. the efficiency of transdifferentiating is 5% or more, 10% or more, 15% or more, 20% or more, or 25% or more;
b. the insulin-producing beta-like cells are maintained after withdrawing the agent;
c. the insulin-producing beta-like cells comprise insulin granules; and/or
d. administration of the agent results in epigenetic changes.

5.-7. (canceled)

8. The method of claim 4, wherein the epigenetic change is a reduction in DNA methylation in the insulin gene locus.

9. A method of treating a subject with diabetes comprising administering an agent that inhibits ALDH3B2 protein or an agent that inhibits the expression of ALDH3B2 gene, wherein the agent increases insulin secretion from the pancreas.

10. The method of claim 9, wherein:
a. the insulin secretion is from pancreatic duct cells or insulin-producing beta-like cells transdifferentiated from pancreatic duct cells;
b. the subject has a blood sugar level higher than 11.1 mmol/liter or 200 mg/dl;
c. the subject has autoimmune diabetes, type 2 diabetes, and/or insulin resistance
d. the subject has lower baseline blood glucose after administration; and/or
the subject has lower blood glucose in response to a glucose challenge after administration.

11. (canceled)

12. (canceled)

13. The method of claim 10, wherein the subject has type 1 diabetes or autoimmune diabetes induced by an immunotherapy.

14.-16. (canceled)

17. A method of preventing the development of diabetes comprising:
a. screening a subject for risk factors for developing diabetes;
b. determining if the subject has increased risk of developing diabetes; and
c. administering an agent that inhibits ALDH3B2 protein or an agent that inhibits the expression of ALDH3B2 gene to a subject that has an increased risk of diabetes.

18. The method of claim 17, wherein screening a subject for risk factors comprises obtaining data on a genetic risk score that is based on the known diabetes-associated gene variants, a family history of diabetes, the presence of one or more autoantibodies against beta cell antigens that are known to predict disease risk, and/or abnormal glucose tolerance.

19. (canceled)

20. The method of claim 9, wherein the subject is human.

21. The method of claim 1, wherein the agent is one or more inhibitor of the ALDH3B2 protein.

22. The method of claim 21, wherein the agent is an ALDH inhibitor.

23. The method of claim 21, wherein the inhibitor is 4-amino-4-methyl-2-pentyne-1-al, benomyl, chloral hydrate, chlorpropamide analogs, citral, coprine, cyanamide, daidzin, 4-(diethylamino)benzaldehyde, disulfiram, gossypol, kynurenine metabolites, molinate, nitroglycerin, and/or pargyline.

24. The method of claim 1, wherein the agent decreases or eliminates expression of the ALDH3B2 protein.

25. The method of claim 24, wherein the agent is a gene editing tool or a small interfering RNA.

26. The method of claim 25, wherein the gene editing tool is a CRISPR/Cas system, transcription activator-like effector nuclease (TALEN), homing endonuclease, or zinc-finger nuclease (ZFN).

27. The method of claim 26, wherein the gene editing tool is delivered by virus transduction or lipid nanoparticle delivery.

28. (canceled)

29. The method of claim 9, wherein the agent is administered in combination with an additional treatment.

30. The method of claim 29, wherein the additional treatment is a glucagon-like peptide analog or agonist, dipeptidyl peptidase-4 inhibitor, amylin analog, biguanide, thiazolidinedione, sulfonylurea, meglitinide, alpha-glucosidase inhibitor, or sodium/glucose transporter 2 inhibitor.
